(19) United States
(12) Patent Application Publication
(10) Pub. No.: US 2002/0072864 A1
(43) Pub. Date: Jun. 13, 2002

(54) COMPUTER-BASED METHOD FOR MACROMOLECULAR ENGINEERING AND DESIGN

(76) Inventors: EMMANUEL LACROIX, LEIMEN (DE); LUIS SERRANO, HEIDELBERG (DE)

Correspondence Address: PENNIE AND EDMONDS, 1155 AVENUE OF THE AMERICAS, NEW YORK, NY, 100362711

(21) Appl. No.: 09/387,741

(22) Filed: Aug. 31, 1999

(51) Int. Cl. G06F 19/00; G01N 33/48

(52) U.S. Cl. 702/27; 702/19

(57) ABSTRACT

The present invention relates to a system and method for engineering and designing a macromolecule. An experimentally determined or de novo atomic structure that corresponds to the macromolecule is identified. The atomic structure is composed of building blocks. When the macromolecule is a peptide or a protein, the building blocks are amino acid residues. A target subset of the building blocks in the atomic structure to be optimized is identified. The coordinates of those building blocks that are not in the target subset are fixed. For each building block in the target subset, a large number of potential conformers is sample d. Each conformer to be sampled is substituted into the atomic structure and tested against an energy function that includes the equivalent energy of the conformer in a reference state. Combinations of conformers that best satisfy an interaction energy function are identified.

### 1. FIELD OF THE INVENTION

The present invention relates to methods for engineering and designing molecules which comprise building blocks that are individually amenable to systematic variation. Particular areas of application include the design and development of macromolecules, for example, proteins, peptides, nucleic acids and polymers with desired properties such as stability and specificity of interaction with counterpart molecules. Specifically, the present invention relates to computer-based methods that employ search methods in the space of available molecules or fragments thereof which could form building blocks of a molecular structure and which use a three dimensional description of the structure with atomic scale resolution. An aim of the invention is to provide guidance to the experimental scientist who is not able to systematically consider all of the possible combinations of building blocks which might need to be permuted.

### 2. BACKGROUND OF THE INVENTION

Biochemistry and synthetic chemistry are replete with molecules whose structures consist of hundreds or thousands of atoms but whose consistency can also be thought of as that of a sequence of identifiable units, each of which comprises only a small number of atoms. Molecules of this sort will herein be referred to as macromolecules, a term which can be taken to include proteins, peptides, cyclic peptides, nucleic acids, lipids, carbohydrates and synthetic polymers, etc. The individual units which go to make them up will be called building blocks, though other terms, both generic and specific, may be found in the art. For example, the building blocks of proteins and peptides are amino acid residues, the building blocks of nucleic acids are nucleotides, and synthetic polymers are built from monomers. The term monomer can also be used in a more general sense. A building block, on its own, will usually differ slightly from its bound form in the macromolecule: the reaction with the building blocks which become its neighbours in the macromolecule structure may truncate its own structure. In this way a different term is often used for the free building block from its bound form. For example, amino acids are the building blocks of peptides and proteins whereas in their bound form they are called residues. The term building block, as used herein, will be understood to mean both the free molecule and its bound form, unless otherwise evident from the context in which it is used. The chemistry and other properties of macromolecules are often understood in terms of the types of building blocks employed and the order in which they are found, i.e., their sequence.

Protein or peptide engineering is a process using recombinant DNA technology or chemical methods to modify the amino acid sequences of natural proteins or peptides to improve or alter their function. By changing, i.e., mutating, the natural amino acid sequence of a protein or peptide, it is possible to alter, inter alia, its stability, substrate specificity, activity, and inter- and intra-molecular interactions. Changes to an amino acid sequence can be made on a purely random basis, or can be derived from educated guesses based on the atomic-resolution detail of the protein or peptide three dimensional structure provided by techniques such as X-ray crystallography, nuclear magnetic resonance, electron microscopy, and electron crystallography.

The mutagenesis of proteins and peptides, even when carried out non-randomly using structural information, can have unexpected or undesired results. First, a mutant protein or peptide may not have any altered characteristics from its native counterpart. Second, a mutant protein or peptide may acquire a completely different set of characteristics from the desired set. Third, a mutant protein or peptide may not be properly folded, rendering it unstable, insoluble, lethal, or completely non-functional. Protein engineering can thus be a trial-and-error process for generating a properly folded mutant protein or peptide with a desired function. Furthermore, mutagenesis to probe the function of proteins, so-called “site-directed mutagenesis”, is a time-consuming process, involving introduction of the mutation into the DNA coding region, transformation of the mutated sequence into the appropriate cells, expression of the protein, purification, and functional assays.

Often, desired changes in protein or peptide function, e.g., altered binding specificity or avidity, require the simultaneous mutagenesis of several amino acids. With twenty possible naturally occurring amino acids at each position, the number of variants that need to be screened is enormous. For changes at three amino acids, there are 8,000 possible combinations; for changes at 10 amino acids, 10¹³ different amino acid sequences are possible. Even though the number of variants may be narrowed by making educated guesses based on knowledge of the protein or peptide structure, a large number of mutants may still have to be made in order to engineer a properly folded protein or peptide with the desired characteristics.

Ab initio peptide and protein design presents more difficulties than the engineering of mutant proteins and peptides. In this case, nearly all of the amino acids must be chosen to create a properly folded peptide or protein with a desired function, making the number of possible variants even greater than for conventional mutagenesis. Furthermore, if the fold of the functional protein or peptide is not well-characterized, or if the structure cannot be designed based on the known structure of an homologous protein (homology modeling), then structural information will not be available to help narrow down those combinations of amino acids that are most likely to adopt the proper protein fold. Therefore, ab initio design of proteins and peptides by in vitro production and testing of all amino acid sequence variants is impractical, if not impossible.

Computer-based methods for designing and engineering proteins and peptides should allow for the identification of amino acid sequence variants that can be accommodated by the three dimensional structure of the protein being mutated, thus decreasing the number of in vitro engineering experiments that need to be performed. The central principle is that it is far easier to consider a large number of sequence variations and choose the best candidates through computer simulation than it is through direct experimentation and synthesis in the laboratory.

Nevertheless, computational methods are non-trivial because of the complexity of the problem and the quality of the primary data that is accessible for immediate use. For example, the high-resolution three dimensional structures of most small organic molecules are available, but those of proteins are typically at lower resolution. Further, the methods of modelling the weak, non-covalent forces, e.g., hydrogen bonds, van der Waals interactions, and hydrophobic interactions, that maintain the three-dimensional structures of macromolecules are at present very crude. And, in general, the number of degrees of conformational freedom that are required to accurately describe the structure of a protein is too large to enable practical exploration of its potential energy surface. Our ability to reliably model small changes in a protein structure is therefore limited by several factors: the accuracy to which the whole structure is known; the impracticality of applying usual optimization methods to systems as large and complicated as proteins; and the inaccuracy of the intermolecular potential functions which are needed to model the ways in which residue side chains determine the three dimensional structure of the protein by aligning with one another.

For these reasons, current computer-based methods for designing and engineering macromolecules cannot efficiently and reliably predict the accommodation of variant structures by an identified protein fold, and thus have limited utility in assessing which sequence variants are likely to have a desired structure and function.

Previous computational approaches to protein engineering have been limited to predictions of tertiary structure from sequence, geometric rather than energetic positioning of side chain atoms, and prediction of favourable sites of cross-linking.

In an analogous way, the exploration of nucleic acid structures is subject to the same complexities of mathematical modelling, as well as the combinatorial problem arising from the fact that at least 4 different nucleotides can be considered for each position on the sequence.

Clearly, there is a need for computer-based methods for designing and engineering macromolecules which utilize a more complete and accurate mathematical description of macromolecular structure than has hitherto been attempted but which employ practical and reasonable approximations to enable efficient execution. In this way it will be possible to predict the structures of macromolecular variants and enable the selection of variants having a desired structure and function.

Citation of a reference herein shall not be construed as indicating that such reference is prior art to the present invention.

### 3. SUMMARY OF THE INVENTION

The present invention relates to an improved computer-based method for optimizing specific building blocks in the sequence set of building blocks which make up a target macromolecule, for example the amino acid residues of a peptide or protein. In essence, the central features of the invention are, given a specification of the building blocks and their desired positions in the macromolecule, use of a plurality of conformations of each building block:; use of a scoring function to quantify and rank the possible structures relative to a reference configuration; and use of filtering techniques for simplifying the analysis. In detail, the method comprises the steps of: (a) specifying at least one substitute for each building block in said set of building blocks; (b) determining, for each substitute, one or more candidate conformers and: (i) substituting the coordinates of each candidate conformer or portion thereof for the corresponding building block or portion thereof in a structure of atomic resolution of said target macromolecule; and (ii) calculating an intrinsic energy term of each candidate conformer; (c) rejecting candidate conformers having an intrinsic energy above a threshold value; (d) calculating a pairwise interaction energy term for all possible pairs of candidate conformers that have not been rejected in step (c) and combining the sum of the pairwise interaction energies for all pairs with the sum of the intrinsic energies for all candidate conformers to give a solution score; (e) determining solutions, from a plurality of solutions, which have, respectively, solution scores that are lower than a predetermined threshold solution score; wherein: (i) each building block in said building block set is represented in each solution in said plurality of solutions by one or more candidate conformers that each correspond to a candidate building block substitute that was independently specified in accordance with step (a) and was not rejected in step (c); and (ii) each solution score representing a difference in the summed potential energy of each candidate conformer in said solution when said candidate conformer is substituted in said atomic-scale resolution structure of the target macromolecule, and when said candidate conformer is substituted into an atomic resolution macromolecular structure corresponding to a reference state. The application of the method may be carried out more than once, sequentially, to obtain better and better solutions. The solutions may then be used as suggestions for synthetic candidates and those molecules which are made may then be assayed against a target of interest.

The present invention further relates to a computer program product for use in conjunction with a computer, the computer program mechanism comprising a computer readable storage medium and a computer program mechanism embedded therein, the computer program mechanism comprising an optimiser module configured to optimize a set of building blocks in a target macromolecule, the optimiser module, (a) upon receiving a request to optimize a set of building blocks and being input at least one substitute for each building block in said set of building blocks; (b) determining, for each substitute, one or more candidate conformers and: (i) substituting the coordinates of each candidate conformer or portion thereof for the coordinates of the corresponding building block or portion thereof in a structure of atomic resolution of said target macromolecule; and (ii) calculating an intrinsic energy term of the candidate conformer; (c) rejecting candidate conformers having an intrinsic energy above a threshold value; (d) calculating a pairwise interaction energy term for all possible candidate conformers that have not been rejected in step (c) and combining the sum of the pairwise interaction energies for all pairs with the sum of the intrinsic energies for all candidate conformers to give a solution score; (e) determining solutions, from a plurality of solutions, which have, respectively, solution scores that are lower than a predetermined threshold solution score; wherein: (i) each building block in said building block set is represented in each solution in said plurality of solutions by one or more candidate conformers that each correspond to a candidate building block substitute that was independently specified in accordance with step (a) and was not rejected in step (c); and (ii) each solution score representing a difference in the summed potential energy of each candidate conformer in said solution when said candidate conformer is substituted in said atomic-scale resolution structure of the target macromolecule, and when said candidate conformer is substituted into an atomic resolution macromolecular structure corresponding to a reference state.

The present invention further comprises a system for optimizing a set of building blocks in a target macromolecule comprising: a central processing unit; an input device for inputting requests; an output device; a memory; at least one bus connecting the central processing unit, and the input device; the memory storing an optimizer module configured to optimize the set of building blocks in the target macromolecule, the optimiser module, (a) upon receiving a request to optimize a set of building blocks and being input at least one substitute for each building block in said set of building blocks; (b) determining, for each substitute, one or more candidate conformers and: (i) substituting the coordinates of the candidate conformer or portion thereof for the coordinates of the corresponding building block or portion thereof in a structure of atomic resolution of said target macromolecule; and (ii) calculating an intrinsic energy term of the candidate conformer; (c) rejecting candidate conformers having an intrinsic energy above a threshold value; (d) calculating a pairwise interaction energy term for all possible candidate conformers that have not been rejected in step (c) and combining the sum of the pairwise interaction energies for all pairs with the sum of the intrinsic energies for all candidate conformers to give a solution score; (e) determining solutions, from a plurality of solutions, which have, respectively, solution scores that are lower than a predetermined threshold solution score; wherein: (i) each building block in said building block set is represented in each solution in said plurality of solutions by one or more candidate conformers that each correspond to a candidate building block substitute that was independently specified in accordance with step (a) and was not rejected in step (c); and (ii) each solution score representing a difference in the summed potential energy of each candidate conformer in said solution when said candidate conformer is substituted in said atomic-scale resolution structure of the target macromolecule, and when said candidate conformer is substituted into an atomic resolution macromolecular structure corresponding to a reference state.

### 4. BRIEF DESCRIPTION OF THE DRAWINGS

FIG. 1: Schematic description of the protein design methods of Perla, the preferred embodiment of the invention.

FIG. 2: Van der Waals interaction energy for two methyl (—CH₃) groups as a function of the distance that separates them.

FIG. 3: Electrostatic interaction between two unit charges of equal sign, either according to Equation V and using a dielectric constant of 4.0_{Γij} (solid line), or according to Equation VI using the same dielectric constant and a screening distance rₛ of 2.0 (dashed line).

FIG. 4: Geometrical conditions to be satisfied in hydrogen bonding.

FIG. 5: Schematic representation of the accessible surface area (ASA) of a protein. This surface is defined as the surface marked by the center of a water molecule (a probe P with radius 1.4 Å) rolling around the protein while maintaining permanent contact with the van der Waals surface of the protein atoms. The arrows indicate that the solvation potential of carbon and oxygen atoms correspond to positive and negative energies, respectively, so that carbon atoms tend to cluster inside the protein while oxygen atoms prefer to protrude outside. The bold surface close to the C and O atoms are their atomic accessible surface areas.

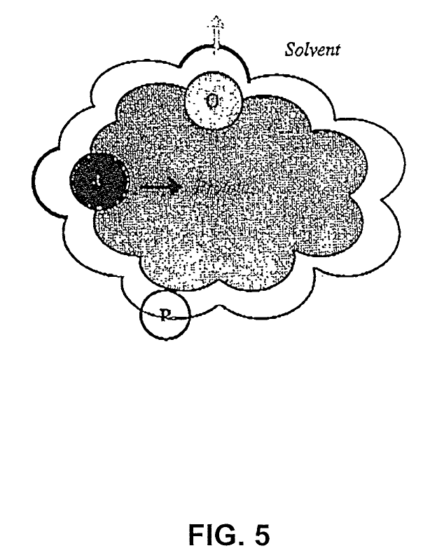

FIG. 6: Conformers generally observed around a rotatable single bond (from left to right: gauche −, trans and gauche +). X and Y represent two heavy atoms, e.g., the alpha and delta carbons of a leucine side chain.

FIG. 7: Distribution of _{χ1} (Val) and _{102 1}, _{χ2} (Leu) dihedral angle values using Gaussian equations (black). Dark grey curves represent the distribution of the trans, gauche − and gauche + conformers. In light grey are the distributions corresponding to non-rotameric configurations.

FIG. 8: Illustration of the rotamer library concept, showing the side chain conformers of valine and leucine. Numbers on top of each structure are the _{102 1} (Val) and _{χ1}, _{χ2} (Leu) dihedral angle values; those labelled with an asterisk were obtained during the evaluation of Perla.

FIG. 9: Block diagram of a system in accordance with the present invention.

FIG. 10: Accompanying the example, distribution of solution scores for 1600 output sequences from Perla, applied to the SH3 domain of alpha-spectrin. Wild Type sequence is indicated along with score of best solution and worst solutions found.

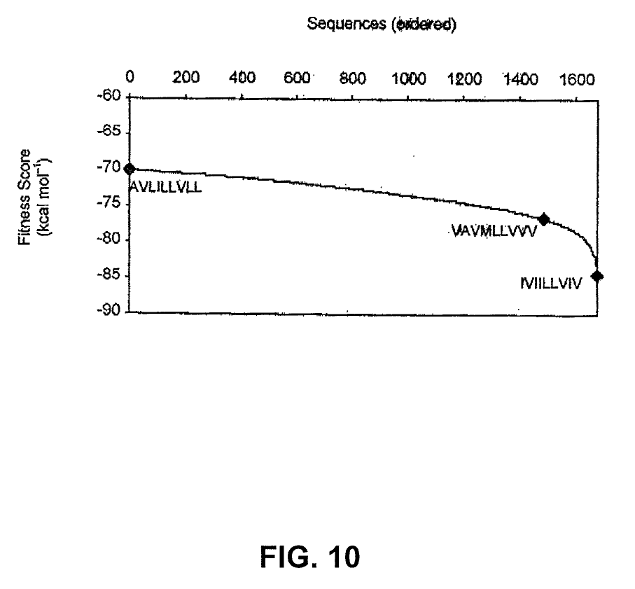

### 5. DETAILED DESCRIPTION OF THE INVENTION

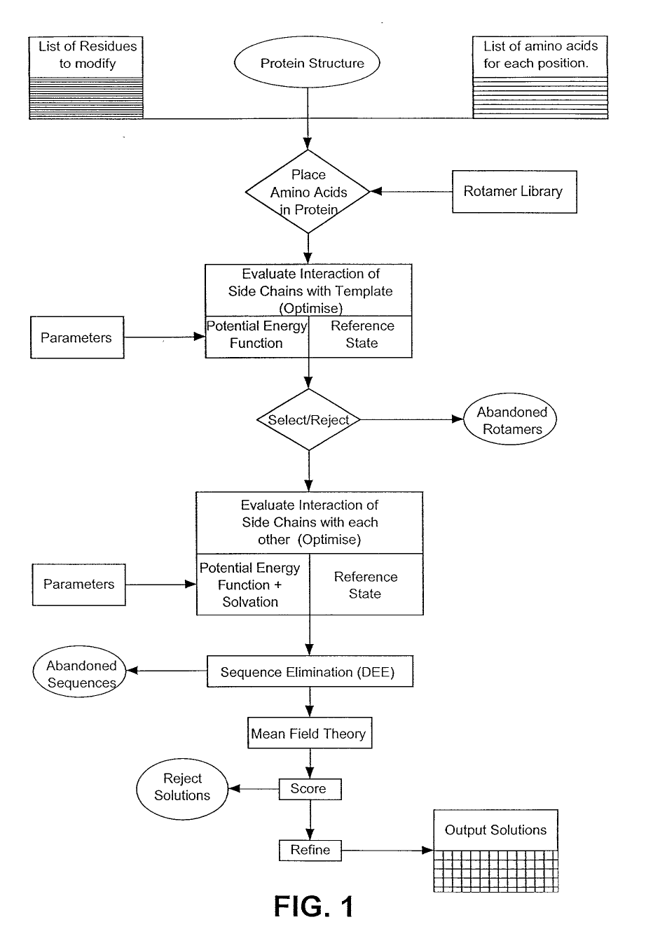

Section 5.1 gives an overview of the invention. In Section 5.2, the method of the present invention as implemented in Perla, the preferred embodiment of the invention, is described in brief (FIG. 1). Subsequent sections describe in more detail each step of the method of the present invention, with emphasis on these steps as implemented in Perla. In Section 5.3, a detailed mathematical description is given of the empirical scoring function used to calculate the energy difference between an optimized conformer of a mutated target protein and some reference state. Section 5.4 provides a detailed theoretical description of the molecular mechanics potential, and of van der Waals, electrostatic, and hydrogen bonding energies, which contribute to it. Section 5.5 provides a detailed mathematical description of the empirical potential, calculated from changes in solvation and entropy of the protein chain, and which introduces an approximate description of the interaction of solvent with the side chain conformers. Section 5.6 describes how the denatured state of proteins are considered in Perla. Section 5.7 describes the generation of the rotamer library used by Perla. Section 5.8 describes energy optimization and elimination of incompatible amino acid conformers. Optimization routines, e.g., dead-end elimination and mean field theory, are detailed in Section 5.9. Section 5.10 describes re-evaluation of solvation energies, and consequently, of the scoring function, for sequences that remain after elimination, and Section 5.11 details output from the preferred embodiment of the present invention. Section 5.12 details a generalisation of the method to macromolecules other than proteins and peptides. Finally, Section 5.13 details a computer system for optimizing a set of building blocks in a macromolecule.

### 5.1. OVERVIEW

The present invention relates to a novel method for designing and engineering macromolecules that utilizes an accurate and complete mathematical representation of macromolecular structure, in order to reliably predict how precise variants of its sequence can be accommodated into a desired three-dimensional (3D) structure. Herein, the 3D structure in question may be a specific conformation of the macromolecule itself or may be a complex in which the macromolecule interacts with a ligand or another macromolecule. A preferred embodiment of the present invention is known as Perla. Perla is a computer-based method for protein engineering that manipulates peptides and proteins in order to identify and sort amino acid sequences capable of folding into a desired 3D structure.

In order to model the effect of a small change on a protein structure, it is not always necessary to reanalyse its entire structure. Consequently, well chosen detailed structural information about the site of mutation may be employed to focus attention on the area of interest. Structural flexibility of a protein may be thought of as a large-scale consequence of the conformational flexibility of the building blocks of which it is composed. Here we may exploit the fact that residue mutation in a protein is effectively a side chain substitution which leaves the backbone unperturbed. That is, the part of the structure of the amino acid building block which changes from one to another is the side chain alone. Correspondingly, conformational analysis may be simplified by using sets of known favourable side chain conformations instead of carrying out an unconstrained energy minimisation.

Furthermore, using the understanding that the three-dimensional structures of proteins are more accurately described as an ensemble of structures in equilibrium that include the active conformation as well as inactive conformations, we may refer energy changes to a denatured state as a reference rather than attempt to model strictly the change in conformation of the folded molecule on altering a side chain.

Although the invention is described herein below mainly with application to proteins and peptides, as will be evident to a skilled artisan, the teaching herein can be readily adapted for use with other macromolecules such as nucleic acids, carbohydrates and other polymers.

### 5.1.1. THE PROTEIN STRUCTURE

The computer-based method of the present invention uses an “inverse folding” approach, i.e., a protein backbone is chosen a priori as the native state to be designed and is kept fixed throughout the calculation. Fixed protein backbone atoms are the central carbon atom, Cα, and the amide group, C(═O)NH, of each residue. There is no restriction that the protein should consist of a single contiguous backbone; Perla can accept multiple backbones as input. The choice of a protein topology depends on the application of the engineered protein. Due to the absence of backbone motions during the evaluation of protein sequences, it is preferable for the main chain target conformation to be correctly constructed from the start. In a preferred embodiment, a protein with a well characterized protein fold or high resolution three dimensional structure is chosen (e.g., from amongst those found in the Protein Data Bank (PDB), available from Research Collaboratory for Structural Bioinformatics (RCSB), web site address http://www.rcsb.org/pdb/). As used herein, the “resolution” of a macromolecular three-dimensional structure is the minimum separation two atoms can have and still appear to be distinct and separate. Thus, the higher the resolution, i.e. the smaller the separation distance at which two atoms can be distinguished, the more accurately determined is the structure. In a preferred embodiment, the protein model is solved at atom level resolution around the site of interest and the fixed backbone has been refined to eliminate steric clashes and unfavourable main chain dihedral angles. For this purpose the structure may have been obtained from X-ray crystallography or from NMR studies. In general, the parameters employed by the user of the invention may be chosen to best suit the quality of the data. In a second embodiment, e.g., de novo protein design, the protein structure is not available or the protein fold is not well characterized, and methods for the construction of novel protein backbones are employed (e.g., WHAT_IF; Vriend, 1990, J. Mol. Graphics 8:52-57; INSIGHT; Abagyan et al., 1994, J. Comp. Chem. 15:488-506).

The 3D structures of other macromolecules can similarly be obtained from X-ray crystallography or may themselves be the outcome of mathematical or computational simulation.

### 5.1.2. THE AMINO ACID SIDE CHAINS

The computer-based method of the present invention uses a three-dimensional atomic description of the system to be engineered. The main chain atomic configuration being provided, the method is used to reconstruct amino acid side chains. The side chains of the twenty naturally occurring amino acid are bound to the backbone Cα atoms.

A custom-made library of discrete side chain conformations (“rotamers”) for each amino acid, compiled using dihedral angle (_{102 1}, _{χ2}, _{102 3}, _{102 4}) data from available structures (preferably from those deposited in the PDB), is employed by the method of the present invention. The library of amino acid side chain conformations is preferably made by fitting occurrences of side chain dihedral angles for each amino acid side chain in known protein structures to Gaussian distributions. Since stereochemical rules were not used to generate the library, it contains less abundant side chain conformations that are nonetheless important components of protein structure. Furthermore, because each dihedral angle is described by a Gaussian distribution, the observed range of oscillation of each angle is also incorporated into the library.

In application to polymeric structures other than proteins, it may be possible to derive conformer libraries from means other than by direct comparison with crystal structures. For example, stereochemical rules may be adequate for the hydroxyl groups of sugar molecules; computer simulation may be most appropriate for the modelling of nucleotide conformations. In some circumstances, the building blocks may have insufficient conformational flexibility to demand construction of conformer libraries. In such cases, the application of the method is a lot more straightforward than described herein, there being fewer conformers per building block.

### 5.1.3. SELECTING POSSIBLE STRUCTURES

The computer-based method of the present invention executes successive trials to consider the immense variety of sequences that can be generated as a result of protein mutagenesis, i.e., substitution of one amino acid side chain with a different amino acid side chain at a given site in the protein.

In one embodiment, the user specifies which residues in the protein are to be altered. To achieve this the user may employ specific knowledge about the 3D structure of the protein. For example, the user may choose residues which seem to be critical to folding or to important in the definition of a binding site. In a preferred embodiment, Perla itself, through use of its own scoring function (see below) may automatically identify the building blocks which are to be varied. In either case, it is not necessary that the selected residues form a contiguous stretch of the sequence of the target macromolecule; nor is it necessary that any pair of the selected residues is adjacent in the sequence.

In one embodiment, the user may also specify a list of possible mutations to be considered at each residue position in the protein or a broad category of desirable mutations. In another embodiment, Perla may analyse the immediate environment of the selected building blocks and choose mutations which are likely to cause the least disruption to that locale. For example, it may be appropriate to consider only “polar” amino acids at a particular position which is already occupied by a polar sidechain.

Sequence sampling as embodied in the method of the present invention consists of searching the required amino acid side chains within the rotamer library and fitting these onto the chosen backbone. Side chains of the amino acid residues that are not mutated can remain structurally fixed or be moved, as desired by the user of the method.

The combinatorial problem of side chain building is solved in the method of the present invention by calculation of mean field energies. This novel integration of Mean Field Theory, an iterative approach, into a protein modeling method provides a measure of the entropy of the molecule and allows for the consideration of all possible amino acid side chain conformations rather than just the global energy minimum, which is a more accurate description of macromolecular structure.

The foregoing steps are applicable to each individual substituted residue; the problem of considering many alternative candidate residues at many different sites is also combinatorial in scope. The invention addresses this aspect by the technique of “dead end elimination” in which certain candidate rotamers may be eliminated from the search space if their energy scores obey certain inequalities with respect to the scores of the other rotamers present in the same solution. Consequently the overall invention comprises two distinct methods of addressing problems of a combinatorial complexity.

### 5.1.4. SCORING THE SOLUTION STRUCTURES

In order to evaluate the degree of fit of a combination of amino acid side chain rotamers to a protein structure, the method of the present invention utilizes a scoring function made up of a sum of terms. Unlike previous methods for protein modeling, the method of the present invention not only considers the global sum of these terms, but also requires that individual terms satisfy constraints found in natural proteins.

Because the nature of the application of the method is to produce a number of different structures, each of which is distinct from the target, it is not possible to meaningfully compare the energies of each. Consequently, the use of a reference structure for each separate solution structure enables the structures to be compared in terms of their inherent stabilities. In a preferred embodiment, in order to assign a score to a particular solution structure, the method calculates the difference in potential energy between the folded protein and the denatured protein.

A preferred embodiment of the present invention calculates a potential energy for the native and denatured (reference) states. For the latter, in a preferred embodiment, sample sequences are taken from structures present in the PDB; this method is described in more detail later. The energy difference between the two states serves as a score, and the higher the score, i.e., the larger the energy difference between the two states, the better the degree of fit of the chosen sequence to the overall native-state protein structure. The potential energies of the native and reference states are partly functions of the atomic configurations of the two structural states. In the rotamer library employed by the methods of the present invention, as in the Protein Data Bank, there are several possible choices for the conformation of each side chain, and therefore, the potential energy of the native state is not a linear function of the amino acid sequence. The potential energy of the reference state is more difficult to quantify, since no single main chain configuration can accurately represent the dynamic ensemble of unfolded structures that comprise the reference state.

The estimation of the native state potential energy requires that the optimal association of amino acid rotamers be found. For peptides longer than a few residues, an exhaustive sampling of every possible combination of rotamers is not practical. Choosing the most likely organization of side chains is a significant combinatorial problem and, therefore, the method of the present invention employs optimization routines. The underlying principle of available optimization methods, e.g., dead-end elimination and mean field theory, is that the energy is expressible as a scoring function (I) comprising a term to describe the fixed template, one sum of terms intrinsic to every single amino acid of the sequence and a second sum for all pairs of residues:

E_{sequence-to-structure}=Eₜₑₘₚₗₐₜₑ+Σ_{all residues}+Σ_{all residue pairs} (I)

In the preferred embodiment of the present invention, a user-defined set of rotatable side chains is modeled in the context of a fixed collection of atoms, which include main 30 chain atoms and the side chain atoms of residues that are not included in the modeling set. Together, the fixed atoms are the template, the structure which is the direct environment of the side chains that are subject to modeling. The calculation of the sequence-independent, constant energy term corresponding to the template (Eₜₑₘₚₗₐₜₑ) is not required for the evaluation of the optimal set of side chains, but can be determined in order to estimate the quality of the template structure itself. Both the intrinsic and pairwise energy terms are similar in nature and are established to correlate with observed structural parameters in proteins. The intrinsic energy term arises from interactions between the (fixed) template and the (rotatable) side chains, while the pairwise energy term arises from interactions of the (rotatable) side chains amongst themselves. Additionally, both the intrinsic and pairwise energy terms contain contributions which depend only on the nature of the residue. A van der Waals component associated with the packing of main chain and side chain atoms, an electrostatic term associated with ion pairs, and a hydrogen bonding term, are contained in both the intrinsic energy term and the pairwise energy term of the scoring function (I). In the preferred embodiment of the present invention, it is not only the global sum of the scoring function that is considered, but also, each individual term must satisfy determined constraints, as reflected in naturally occurring protein structures.

In other embodiments of the present invention and in application to macromolecules other than proteins, it may be preferable to use more than one reference state for the calculation of the scoring function. Alternatively, in other embodiments the use of a reference state may be unnecessary.

In yet other embodiments, the scoring function may comprise a term quantifying the interaction between the macromolecule and some binding partner. For example, the macromolecule may be an enzyme and the partner its substrate; in another example, the macromolecule may be a peptide sequence and its partner may be another peptide sequence; in a further example, the macromolecule may be a nucleic acid and its partner may be a protein or some fragment thereof.

### 5.2. THE STEPS OF SEQUENCE MODELING AND EVALUATION USING PERLA

Perla, the preferred embodiment of the present invention, first reads the user-specified input which consists of three pieces of information. (a) the atoms comprising the specified template (or “target”) protein conformation and their Cartesian coordinates. These coordinates may have originated as fractional coordinates from a Protein Data Bank (PDB) file. There is no restriction that the atoms comprising the template form a connected unit, i.e., the protein may have multiple backbones, or several discrete proteins or peptides in juxtaposition may constitute the template. It is preferred to utilise other computational tools which ascertain the appropriate protonation state of the residues and ionise them as applicable, before passing the coordinates to Perla. (b) a selection of amino acids to engineer at determined positions, or an indication that Perla should make some determinations of its own in this regard, and (c) a series of adjustable input parameters that set weights for the different energy terms, place thresholds and penalties to control the flow of output and tune the effectiveness of the optimization procedure. In situations where the residues to be modified are close to one another, Perla can automatically determine which residues in the vicinity should also be subject to optimization of side chain conformations.

Side chains that correspond to the list of amino acids to model are obtained from a rotamer library and their interaction with the template protein conformation is computed, i.e., the intrinsic energy term of the scoring function (I) is determined by summing and optimizing van der Waals, electrostatic, and hydrogen bonding energies and then adding backbone entropy costs. Side chain conformers that are not compatible with the template structure are eliminated.

Subsequently, pairs of rotamers are considered in order to evaluate the pairwise (side chain-side chain) component of the scoring function (I). Again, van der Waals, electrostatic, and hydrogen bonding energies are summed and optimized, and then a pairwise solvation contribution is added. No elimination is performed, since the identification of an energetically disfavored pair does not imply that the participating side chains are incompatible with the target protein fold.

In order to reduce the number of sequences to sample and to ultimately find that which has the optimal sequence-to-structure relationship, e.g., the lowest native state potential energy (lowest score) or the greatest energy difference in energy from the reference state, dead-end elimination is used to mark and discard sequences that cannot achieve the energy minimum.

For all remaining sequence combinations, mean field theory enables the estimation of weights for all side chain rotamers, which are then used to compute the score of each sequence. Sequences that do not score well are rejected, while for others, the solvation term is reevaluated. Some sequences may be eliminated at this step if they have poor salvation.

Finally, in the output from the program, the engineered sequences are accompanied by a description of the energy terms that contribute to the scoring function and a set of three-dimensional Cartesian coordinates that describe the modeled structure.

Those skilled in the art will recognize that, although the set of parameters used by the method of the present invention relate to amino acids, corresponding parameters for nucleic acids and other organic compounds, e.g., carbohydrates, are available and can readily be integrated into the method as applied to other macromolecules.

### 5.3. THE SCORING FUNCTION

Central to the operation of Perla is the use of an empirical scoring function to calculate the energy difference between an optimized conformer of a mutated version of the target protein and some corresponding reference state. The way in which the reference state is constructed is for proteins is described in more detail below, section 5.6.

The context in which the scoring function should be viewed is that the protein comprises a fixed backbone (or backbones) of amino acid residues, some specified subset of which are to be varied. The backbone and the constant residues (including their side chains) together form the template. The side chains of the variable residues interact with the template, giving rise to the “intrinsic” energy term and amongst themselves, giving rise to the “pairwise” energy term. As previously described, in the preferred embodiment of this invention, the scoring function is therefore decomposed into a sum of terms, described respectively as “template”, “intrinsic” and “pairwise”. As will be shown, each of these terms partitions into summed contributions. The contributions are regarded to be either components of a molecular mechanics model or part of an empirical description of solvation and entropic factors. The theory behind each of these categories is described later in this section.

In other embodiments of the present invention, the scoring function may comprise pairwise terms in which interactions between atoms on the macromolecule and atoms on some binding partner of interest are computed.

### 5.3.1. THE TEMPLATE TERM

The template energy term consists of 6 contributions:

$${E}_{Template} = {E}_{Template}^{Molecular Mechanics} + {E}_{Main Chain}^{Entropy } + {E}_{Side Chain}^{Entropy} + {E}_{Side Chain}^{Vibrational Entropy} + {E}_{Template}^{Solvation} + {E}_{Residues}^{Statistical Penalty}$$

In this expression, the subscript “Template” means all atoms of the template whereas the other subscripts identify particular categories of atoms. The last term is related to the identity of the amino acid residues in the sequence. Each of the components in the expression includes as a coefficient, a user-defined weighting factor, ω, which can be adjusted to suit different applications.

The explicit form of the terms is as follows. The molecular mechanics terms describe long-range interactions between pairs of atoms in the template and supply the difference in such terms between the target protein and the reference state.

$${w}^{vdw} [ \begin{matrix}{\sum }_{non \text{-} bonded atoms i , j} {( \frac{{A}_{ij}^{vdw}}{{r}_{ij}^{12}} - \frac{{B}_{ij}^{vdw}}{{r}_{ij}^{6}} )}_{\begin{matrix}target \\ structure\end{matrix}} - \\ {\sum }_{non \text{-} bonded atoms i , j} {( \frac{{A}_{ij}^{vdw}}{{r}_{ij}^{12}} - \frac{{B}_{ij}^{vdw}}{{r}_{ij}^{6}} )}_{\begin{matrix}reference \\ frame\end{matrix}}\end{matrix} ] + {w}^{hb} [ \begin{matrix}{\sum }_{H \text{-} bonded atoms H , A} {( \frac{{A}_{HA}^{hb}}{{r}_{HA}^{12}} - \frac{{B}_{HA}^{hb}}{{r}_{HA}^{10}} )}_{\begin{matrix}target \\ structure\end{matrix}} - \\ {\sum }_{H \text{-} bonded atoms i , j} {( \frac{{A}_{HA}^{hb}}{{r}_{HA}^{12}} - \frac{{B}_{HA}^{hb}}{{r}_{HA}^{10}} )}_{\begin{matrix}reference \\ name\end{matrix}}\end{matrix} ] + {w}^{elec} [ \begin{matrix}{\sum }_{atomic charges} {( \frac{{q}_{i} {q}_{j} {e}^{2}}{4 π {ϵ}_{0} {r}_{ij}} )}_{\begin{matrix}target \\ structure\end{matrix}} - \\ {\sum }_{atomic charges} {( \frac{{q}_{i} {q}_{j} {e}^{2}}{4 π {ϵ}_{0} {r}_{ij}} )}_{\begin{matrix}reference \\ name\end{matrix}}\end{matrix} ]$$

The second term describes the entropy cost of fixing the main chain at the physiological temperature, T_{phy},

$$- {w}_{mainchain}^{entropy} {RT}_{phys} {\sum }_{all residuesi} ln \frac{{\sum }_{\begin{matrix}subspaces {φψ}_{20 ° \times 20 °} \\ close to {φ}_{i} {ψ}_{i}\end{matrix}} {w}_{{φψ}_{20 ° \times 20 °}} {N}_{{φψ}_{20 ° \times 20 °}}^{amino acid i}}{{N}_{all φψ}^{amino acid i}}$$

The third term represents the entropy cost of placing amino acid side chains into the template structure where they are more hindered due to the compact protein environment. This term is, again, a difference between the entropies of the side chains in the template and the reference.

$$- {w}_{sidechain}^{vibration} {T}_{phys} {\sum }_{all residues i} [ \begin{matrix}{( - R {\sum }_{\begin{matrix}all residues r \\ all residue i\end{matrix}} {w}_{r} ln {w}_{r} )}_{\begin{matrix}target \\ structure\end{matrix}} - \\ {( - R {\sum }_{\begin{matrix}all sub \text{-} rotamers r \\ of residue i\end{matrix}} {w}_{r} ln {w}_{r} )}_{\begin{matrix}reference \\ frame\end{matrix}}\end{matrix} ]$$

The fourth term represents the entropy cost of restricting the “vibrational” freedom of rotamers. It allows priority to be given to side chain rotamers that can freely rotate within a space corresponding to the Gaussian distributions determined during the creation of the rotamer library.

$$- {w}_{side chain}^{vibration} {T}_{phys} {\sum }_{all residues i} {\sum }_{\begin{matrix}all rotamers r \\ of residues i\end{matrix}} {w}_{i} [ \begin{matrix}{( - R {\sum }_{\begin{matrix}all sub \text{-} rotamers s \\ of rotamer r\end{matrix}} {w}_{s} ln {w}_{s} )}_{\begin{matrix}target \\ structure\end{matrix}} - \\ {( - R {\sum }_{\begin{matrix}all sub \text{-} rotamers s \\ of rotamer r\end{matrix}} {w}_{s} ln {w}_{s} )}_{\begin{matrix}reference \\ frame\end{matrix} }\end{matrix} ]$$

The fifth term is the solvation energy, computed as a difference in the energy of interaction between the target structure and surrounding solvent and the reference structure and surrounding solvent.

$$+ {w}^{solvation} [ {( {\sum }_{atoms i} {σ}_{i} {ASA}_{i} )}_{\begin{matrix}target \\ structure\end{matrix}} - {( {\sum }_{atoms i} {σ}_{i} {ASA}_{i} )}_{\begin{matrix}reference \\ frame\end{matrix}} ]$$

The last term is a statistical penalty function which is introduced to drive the sequence design towards a sequence subspace known a priori to be plausible.

$$- {w}^{stat} {RT}_{stat} {\sum }_{all residues i} ln {P}_{amino acid i}^{stat}$$

For example, the parameters of this term can represent amino acid relative abundance in the protein database or in sequence alignment related to the a family of proteins containing the target protein. The effective temperature, Tₛₜₐₜ, associated with this term, might differ from the actual physical temperature T_{phys} used for entropy related terms.

### 5.3.2. THE INTRINSIC TERM

The computational partitioning of the terms arises now because both dead-end elimination and mean field approximation routines work only with a set of pairwise descriptors of the energy. Hence the invention provides an intrinsic energy term for all candidate rotamers, that represents the interaction of each with the main chain (and any other side chain that is kept fixed).

The intrinsic energy term consists of 5 contributions, each pertaining to interactions of the side chains of the variable residues with the template.

$${E}_{Intrinsic} = {E}_{Side Chain - Main Chain}^{Molecular Mechanics} + {E}_{Main Chain}^{Entropy } + {E}_{Side Chain}^{Vibrational Entropy} + {E}_{SideChain}^{Solvation} + {E}_{Residues}^{Statistical Penalty}$$

These terms mirror those in the template term. The molecular mechanics term is as follows.

$$\{ \begin{matrix}{w}_{Intrinsic}^{vdw} [ {\sum }_{\begin{matrix}non \text{-} bonded \\ atoms i of side chain \\ and j of main chain\end{matrix}} {( \frac{{A}_{ij}^{vdw}}{{r}_{ij}^{12}} - \frac{{B}_{ij}^{vdw}}{{r}_{ij}^{6}} )}_{\begin{matrix}target \\ structure\end{matrix}} - {VDW}_{\begin{matrix}reference \\ frame\end{matrix}}^{\begin{matrix}residue \\ type\end{matrix}} ] + \\ {w}_{Intrinsic}^{hb} [ {\sum }_{\begin{matrix}H \text{-} bonded \\ atoms H or A of side chain \\ and H or A of main chain\end{matrix}} {( \frac{{A}_{HA}^{hb}}{{r}_{HA}^{12}} - \frac{{B}_{HA}^{hb}}{{r}_{HA}^{10}} )}_{\begin{matrix}target \\ structure\end{matrix}} - {HB}_{\begin{matrix}reference \\ frame\end{matrix}}^{\begin{matrix}residue \\ type\end{matrix}} ] + \\ {w}_{Intrinsic}^{ele} [ {\sum }_{\begin{matrix}atomic \\ charges i of side chain \\ and j of main chain\end{matrix}} {( \frac{{q}_{i} {q}_{j} {e}^{2}}{4 π {ϵ}_{o} {ϵ}_{r} {r}_{ij}} )}_{\begin{matrix}target \\ structure\end{matrix}} - {ELE}_{\begin{matrix}reference \\ frame\end{matrix}}^{\begin{matrix}residue \\ type\end{matrix}} ]\end{matrix}$$

The portion of the energy measured in the reference frame is dependent only on the amino acid type, not its geometry, and thus is the same for each rotamer. The role of this term is to help determine which sequences might be poor quality and not to distinguish between rotamer combinations of a particular sequence. The parameters that are used in the molecular mechanics term are derived from calculations done with Perla over a large sample of main chain structures and sequences, whose results are averaged.

Second, the main chain entropy cost is also completely independent of the rotamer configuration and thus is an aid to distinguishing between sequences only.

$$- {w}_{mainchain}^{entropy} {RT}_{phys} ln \frac{{\sum }_{\begin{matrix}subspaces {φψ}_{20 ° \times 20 °} \\ close to {φ}_{i} {ψ}_{i}\end{matrix}} {w}_{{φψ}_{20 ° \times 20 °}} {N}_{{φψ}_{20 ° \times 20 °}}^{\begin{matrix}residue \\ type\end{matrix}}}{{N}_{all φψ}^{\begin{matrix}residue \\ type\end{matrix}}}$$

The third term, for describing the side chain rotamer vibration entropy cost is measured with respect to a set of tabulated references, which are currently derived from a uniform distribution. The weights of the sub-rotamers are obtained from the partition function computed to reject the least probable rotamers (see section 5.9 for details).

$$- {w}_{side chain}^{vibration} {T}_{phys} [ {( - R {\sum }_{sub \text{-} rotamer s} {w}_{s} ln {w}_{s} )}_{\begin{matrix}target \\ structure\end{matrix}} - {VIB}_{\begin{matrix}reference \\ frame\end{matrix}}^{{residue}_{type}} ]$$

The fourth term which measures the solvation energy has been obtained by cutting the surface areas into intrinsic and pairwise parts.

$$+ {w}^{solvation} {\sum }_{atoms i of side chain} {σ}_{i} [ {( {ASA}_{i} )}_{\begin{matrix}reference \\ frame \end{matrix}} - {( {ASA}_{i} )}_{\begin{matrix}target \\ structure\end{matrix}} ]$$

The solvation term is expressed from the relative buried surface area rather than the exposed surface area (thus the invention provides a subtraction in the sense of reference-target and not target-reference). For this reason, a different set of solvation parameters is used, as described later.

Finally, the fifth term is once again a statistical contribution, which should consist of tabulated values or probabilities given by the user in a readable format.

$$- {w}_{Intrinsic}^{stat} {RT}_{stat} ln {P}_{\begin{matrix}residue \\ type\end{matrix}}^{stat}$$

### 5.3.3. THE PAIRWISE TERM

Finally, the pairwise energy term represents the interaction energy of a pair of candidate rotamers and is therefore summed over all pairs of candidate rotamers. The previous comments about the reference states and temperatures are applicable here.

The pairwise term comprises 4 contributions:

$${E}_{Intrinsic} = {E}_{Side Chain - Side Chain}^{Molecular Mechanics} + {E}_{Side Chain}^{Vibrational Entropy} + {E}_{SideChain}^{Solvation} + {E}_{Residues}^{Statistical Penalty}$$

Similarly to the Intrinsic term, the molecular mechanics term contains reference state terms which depend only on amino acid composition. The summations run over pairs of atoms on different residue side chains.

$$\{ \begin{matrix}{w}_{Intrinsic}^{vdw} [ {\sum }_{\begin{matrix}non \text{-} bonded \\ atoms i of side chain \\ and j of main chain\end{matrix}} {( \frac{{A}_{ij}^{vdw}}{{r}_{ij}^{12}} - \frac{{B}_{ij}^{vdw}}{{r}_{ij}^{6}} )}_{\begin{matrix}target \\ structure\end{matrix}} - {VDW}_{\begin{matrix}reference \\ frame\end{matrix}}^{\begin{matrix}residue \\ type\end{matrix}} ] + \\ {w}_{Intrinsic}^{hb} [ {\sum }_{\begin{matrix}H \text{-} bonded \\ atoms H or A of side chain \\ and H or A of main chain\end{matrix}} {( \frac{{A}_{HA}^{hb}}{{r}_{HA}^{12}} - \frac{{B}_{HA}^{hb}}{{r}_{HA}^{10}} )}_{\begin{matrix}target \\ structure\end{matrix}} - {HB}_{\begin{matrix}reference \\ frame\end{matrix}}^{\begin{matrix}residue \\ type\end{matrix}} ] + \\ {w}_{Intrinsic}^{ele} [ {\sum }_{\begin{matrix}atomic \\ charges i of side chain \\ and j of main chain\end{matrix}} {( \frac{{q}_{i} {q}_{j} {e}^{2}}{4 π {ϵ}_{o} {ϵ}_{r} {r}_{ij}} )}_{\begin{matrix}target \\ structure\end{matrix}} - {ELE}_{\begin{matrix}reference \\ frame\end{matrix}}^{\begin{matrix}residue \\ type\end{matrix}} ]\end{matrix}$$

The second term, for the rotamer vibration entropy is formulated to measure the change of entropy due to the interaction of the two side chain rotamers taking as a reference the vibration entropy of each rotamer substituted separately in the target structure.

$$- {w}_{Pairwise}^{vibration} {T}_{phys} λ [ \begin{matrix}{( - R {\sum }_{\begin{matrix}sub \text{-} rotamers {A}_{s} and {B}_{s} \\ of rotamer pair AB\end{matrix}} {w}_{{A}_{s} {B}_{s}} ln {w}_{{A}_{s} {B}_{s}} )}_{\begin{matrix}rotamer pair AB \\ in target structure\end{matrix}} - \\ {( - R {\sum }_{\begin{matrix}sub \text{-} rotamers {A}_{s} \\ of rotamer A\end{matrix}} {w}_{{A}_{s}} ln {w}_{{A}_{s}} )}_{\begin{matrix}only rotamer A \\ in target structure\end{matrix}} - \\ {( - R {\sum }_{\begin{matrix}sub \text{-} rotamers {B}_{x} \\ of rotamer B\end{matrix}} {w}_{{B}_{s}} ln {w}_{{B}_{s}} )}_{\begin{matrix}only rotamer B \\ in target structure\end{matrix}}\end{matrix} ]$$

This entropy term is scaled by a factor λ to avoid an overestimation when summing over all pairs of interacting rotamers (see section 5.7 for details).

The third term, for the difference between accessible surface areas is formulated to measure the area buried between the two side chains and that the solvation term is now also scaled by a factor λ to avoid an overestimation of the buried surface areas (likely to be counted several times when summing over all pairs of interacting rotamers).

$$+ {w}^{solvation} {\sum }_{\begin{matrix}atoms i of residue A or B \\ of residue pair AB\end{matrix}} {σ}_{i} λ [ \begin{matrix}{( {ASA}_{i} )}_{\begin{matrix}only residue A or B \\ in target structure\end{matrix}} \\ - {( {ASA}_{i} )}_{\begin{matrix}only residue AB \\ in target structure\end{matrix}}\end{matrix} ]$$

The final term is, as previously, introduced to bias against improbable sequences of residues.

$$- {w}_{Pairwise}^{stat} {RT}_{stat} ln {P}_{\begin{matrix}residue \\ pair\end{matrix}}^{state}$$

We note that the entropy of the side chains is dependent upon the weight distribution calculated by the mean field approximation routine (section 5.9). Hence, that part of the energy is not included at all in either the intrinsic or pairwise description. By contrast, the vibration entropy cost is used to penalize rotamers whose interaction energy (either intrinsic or pairwise) is only optimal for a few of the sub-rotamer conformations they can adopt (see section 5.7).

### 5 5.4. THE MOLECULAR MECHANICS POTENTIAL

In the method of the present invention, a protein is represented as an ensemble of atoms with discrete masses and partial charges, and therefore, classical mechanics equations are applied to estimate the potential energy of the system.

### 5.4.1. THE BOND STRETCH AND BOND-ANGLE TERMS

The standard molecular mechanics function (or “force field”) is a sum of terms that are related to bonded or nonbonded interactions and that depend on the atomic configuration, which is described by the coordinate vectors, rᵢ (for an overview, see van Gunsteren & Berendsen, 1990, Angew. Chem. Int., Ed. Engl. 29:992-1023):

$$V ( {r}_{1} , … , {r}_{n} ) = {\sum }_{bonds} \frac{1}{2} {{K}_{b} ( b - {b}_{0} )}^{2} + {\sum }_{angles} \frac{1}{2} {{K}_{φ} ( φ - {φ}_{0} )}^{2} + {\sum }_{dihedrals} {K}_{ϕ} [ 1 + cos ( n ϕ - δ ) ] + {\sum }_{nonbonded i , j} ( {A}_{ij} / {r}_{ij}^{12} - {B}_{ij} / {r}_{ij}^{6} ) + {\sum }_{H \text{-} bonds i , j} ( {A}_{ij}^{r} / {r}_{ij}^{12} - {B}_{ij}^{′} / {r}_{ij}^{10} ) + {\sum }_{charges i , j} {q}_{i} {q}_{j} {e}^{2} / 4 π {ϵ}_{0} {ϵ}_{r} {r}_{ij}$$

Molecular mechanics is a well-established sphere of research and several widely used implementations exist: for example, AMBER, CHARMM, ECEPP2, MM2, CVFF, all of which are commercially or freely available. The operation of Perla is not dependent on the specific force-field which is used.

The first three terms of the molecular mechanics force field correspond to bonded interactions. The first represents the elongation of covalent bonds between two atoms (bond stretching). It has a harmonic form, where b is the effective bond length, b₀ is the ideal length (energy minimum), and K_{b} is the force constant that is characteristic of the actual type of covalent bond. The second term similarly describes the deformation of the angle φ formed by three covalently bonded atoms (bond-angle bending). The third accounts for the rotation around bonds, or dihedral angles φ, according to a periodic potential with phase δ.

The description of side chain conformations as a set of rotamers consists of setting the corresponding dihedral angles at values corresponding to energy minima. In addition, the covalent bond lengths and angles are set to their ideal values and are invariant. Therefore, the related energy terms are neglected and the methods of the present invention only consider the remaining three terms: van der Waals, hydrogen bonding and electrostatic interactions. These noncovalent forces that maintain protein three-dimensional structures, are the most important for a valid representation of protein structure, and are described in mathematical detail below. In a preferred embodiment, all parameters, e.g., atomic charges and van der Waals energy parameters, are taken from the ECEPP/2 potential (Momani et al., 1975, J. Phys. Chem. 79:2361-2381; Nemethy et al., 1983, J. Phys. Chem. 87:1883-1887).

### 5.4.2. THE VAN DER WAALS ENERGY

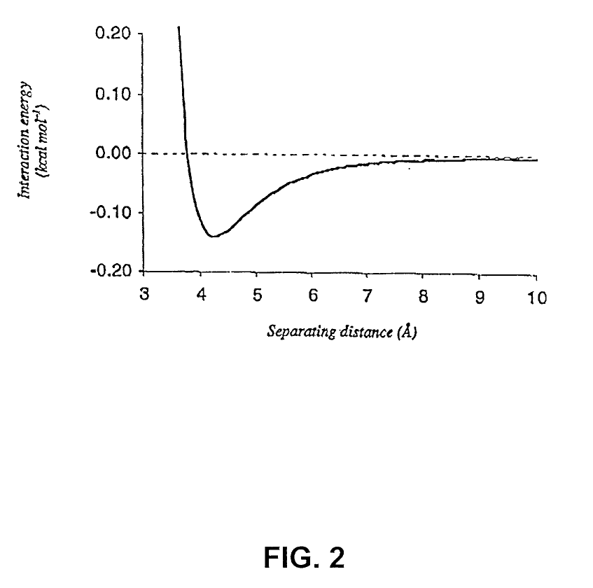

Van der Waals interactions originate from a nonspecific attractive force that exists between atoms. That force is due to the transient asymmetry of the distribution of electronic charge around an atom, which induces a similar asymmetry in the distribution of electronic charge around neighboring atoms. The attraction increases as the distance between atoms decreases, until it is at a maximum when the two atoms, i and j, are separated by a distance rᵢⱼ, which is about 0.3-0.5 Å larger than the van der Waals contact distance, (the closest contact distance between the two atoms that is observed in crystal structures). The overlapping of the electron clouds of atoms i and j creates strong dominant repulsions at shorter distances (FIG. 2).

The van der Waals interaction energy between two atoms i and j can be described by a standard 6-12 Lennard-Jones potential, and the total van der Waals interaction energy term is the sum of the interaction energies between all nonbonded atom pairs:

E_{vdw}Σ_{nonbonded ij}(Aᵢⱼ/rᵢⱼ¹²−Bᵢⱼ/rᵢⱼ⁶)

where Γᵢⱼ is the distance separating atoms i and j, and Aᵢⱼ and Bᵢⱼ are related to the chemical nature of the interacting atoms. Thus, the energy term consists of a repulsive part that decays with r¹², and an attractive part that varies inversely with r⁶.

Van der Waals energies for pairs of atoms are on the order of the average thermal energy of molecules at room temperature (˜0.6 kcal/mol) and diminish rapidly even for a small increase of interatomic distances. Thus, the van der Waals interaction becomes significant only when many interacting pairs form simultaneously, such as in the folded state of a protein. Most importantly, van der Waals energies are critical probes of the packing quality within the three-dimensional fold. For any sequence to fit a given fold, steric compatibility is required and no positive van der Waals energies should be tolerated. Cavities, which produce van der Waals energies near zero, should also be avoided, especially if they are small enough to exclude solvent water molecules.

In the reference (denatured) state, atomic contacts within the polypeptide are less common and intra-molecular van der Waals interactions are not as significant as in the folded state, since van der Waals contacts with the solvent partly compensate for the loss of interaction. Therefore, most existing computer-based methods for designing proteins neglect the van der Waals contribution of the denatured state.

However, consideration of the van der Waals energy of the reference state of a protein in the method of the present invention results in the removal of scaling artefacts in the van der Waals energy term. Potential energy functions that sum over all atoms of a system scale with the number of interacting atoms, resulting in scaling artefacts that should be removed from energy terms that result in large numbers. In the method of the present invention, scaling artefacts are avoided when comparing two sequences with different amino acid compositions by referencing each sequence to a denatured conformation. In the preferred embodiment of the present invention, van der Waals reference energies for each amino acid are utilized. These may be obtained in several different ways. In one approach, energy terms were calculated for each of the twenty amino acids in an extended five-residue peptide with alanine residues flanking the residue of interest. In another approach, energy terms are calculated for each of the amino acids, as found in a population of protein fragments similar to the population of unfolded structures. The reference energies scale with the number of atoms in each amino acid and compensate for the larger van der Waals contribution of larger residues in folded proteins.

### 5.4.3. THE ELECTROSTATIC ENERGY

The interaction energy between electrostatic charges is fundamental and is described as the sum over all nonbonded atoms i and j, as follows:

EₑₗₑΣ_{charges i,j}qᵢqⱼe²/4πε₀εᵣrᵢⱼ

wherein qᵢ and qⱼ are the numbers of charges on atoms i and j, respectively, rᵢⱼ is the distance between the two atoms, e is the charge of an electron, and ε₀ and εᵣ are the permittivity of the vacuum and the medium relative dielectric constant, respectively.

In a vacuum, the electrostatic potential of an atomic charge in the field of another is the product of the two atomic charges divided by the distance that separates them (from Coulomb's Law). For two charges of opposite sign, the energy decreases as the atoms approach each other; the energy increases with decreasing distance if the charges have the same sign. The interaction is strong, e.g., up to 100 kcal/mol, and is effective over large distances.

In media other than vacuum, the strength of the interaction is significantly reduced to less than several kcal/mol by the relative dielectric constant εᵣ. The εᵣ of water has a value of about 80; the interior of a protein, which is mainly packed with carbon atoms, is less polar and has a lower dielectric constant, usually 4.0.

In the method of the present invention, two dielectric constants (one for water or bulk solvent and one for the interior of the protein) are used for each mutated residue, according to the degree of burial of the side chain at the related position in the target protein structure. Every side chain atom is determined to be “exposed” or “buried” according to some geometric criterion. In one embodiment, this criterion is derived from the relative proportion of the solvent accessible surface area of the side chain that is assigned to the atom. In a preferred embodiment, the geometric description takes into account the distance from Cα to the nearest solvent molecule, taken along the Cα-Cβ vector. For each pair of atoms for which an electrostatic interaction is being calculated, the dielectric constant used will be the solvent value if both atoms are exposed, the protein interior value if both atoms are buried. When the interaction is between a completely buried atom and a completely exposed atom, the average of both dielectric constants is used.

In addition, the electrostatic energy term is modified to lessen the importance of the interaction at long distance. In one embodiment, shown in Equation II, dielectric constants are scaled linearly with the separation distance between atoms i and j:

Eₑₗₑ=Σ_{charges i,j}(qᵢqⱼe²/4πε₀rᵢⱼ) (1/εᵣrᵢⱼ) (II)

In the preferred embodiment of the present invention, the pH is considered to be neutral, and the parameters used are for fully charged versions of acidic (aspartic acid, pK=3.5; glutamic acid, pK=4.5; histidine, pK=6) and basic (lysine, pK=11; arginine, pK=12) amino acids. Therefore, the entire electrostatic energy term is scaled by an exponential factor to account for the screening of charges by salts and counterions, as shown in Equation III:

Eₑₗₑ=Σ_{charges i,j}(qᵢqⱼe²/4πε₀r_{y}) (exp−r_{y}/rₛ) (III)

The rate of exponential damping is controlled by a decay constant, rₛ, whose units are those of distance.

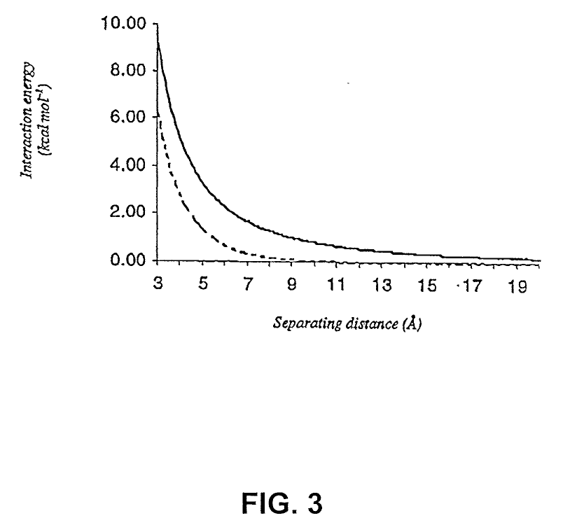

FIG. 3 illustrates the electrostatic interaction energy between two unit charges of equal sign as a function of interatomic separation calculated using either Equation II or Equation III.

In the denatured state, electrostatic energy terms contribute less to the potential energy due to the overall increase in interatomic distances caused by extension of the protein chain, and more importantly, to higher solvation and charge screening. In contrast, in the folded protein, amino acids separated by only a few residues rarely undergo a conformational change that gives rise to a significant change in the distances separating their atomic charges. Therefore, if only the native state of the protein is considered, the electrostatic term is over-estimated.

In one embodiment of the present invention, values are tabulated to represent all possible electrostatic interactions in the denatured state as a function of the sequence separation. In the preferred embodiment of the present invention, the electrostatic energy term of the reference state is zero.

### 5.4.4. HYDROGEN BONDING ENERGY

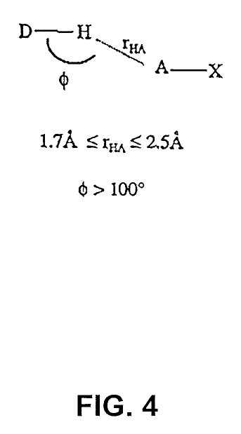

A hydrogen bond is formed when two electronegative atoms, a donor and an acceptor, compete for the same hydrogen atom. As a result, the distance between the hydrogen atom of the hydrogen bond donor and the hydrogen bond acceptor is shorter than the van der Waals contact distance, although it is larger than the length of a covalent bond. The interaction is partly covalent and partly electrostatic in nature and can have an energy of up to 7 kcal/mol. Hydrogen bonds are highly directional and occur predominantly with the donor, hydrogen, and acceptor in a collinear orientation. Therefore, the potential energy function of the preferred embodiment of the present invention considers hydrogen bonding only if the geometrical conditions are satisfied, i.e., if the distance between the hydrogen and the acceptor atom is between 1.7 Å and 2.5 Å, and the angle made by the donor, hydrogen and acceptor is greater than 100° (FIG. 4). If these conditions are met, a hydrogen bonding term (Equation IV) replaces the van der Waals term corresponding to the interaction between the hydrogen and acceptor atoms.

E_{HB}=Σ_{H-bonded H,A}(A_{HA}/rᵢⱼ¹²−B_{HA}/rᵢⱼ¹⁰) (IV)

The preferred embodiment of the present invention does not take into account the possibility that there is intra-molecular hydrogen bonding in the denatured state, since the geometrical conditions are only fulfilled if elements of structure, e.g. turns or a-helices, form locally. The formation of hydrogen bonds between atoms in the denatured protein and water is included empirically in an accessible surface-area-dependent solvation potential described below in Section 5.5. In essence, the residues in a denatured protein are modelled by ensembles of representative fragments taken from protein structures in the PDB.

### 5.5. THE EMPIRICAL POTENTIAL

The method of the present invention, as implemented in the preferred embodiment, Perla, also evaluate changes in entropy and solvation of the protein chain by means of empirical models constructed to account for properties that cannot be broken down into a set of well characterized physical forces. It is customarily difficult to accurately model entropy and solvation, because a practical and accurate representation of the ensemble of unfolded protein structures would have to be developed. This would necessitate the handling of an enormous number of either water molecules or chain configurations using a reasonable amount of computing time, as well as the development of an accurate set of energy parameters to describe these unfolded states. Instead Perla adopts pragmatic levels of approximation.

### 5.5.1. SOLVATION

Proteins function in aqueous media, which are poor solvents for apolar molecules because apolar molecules cannot participate in hydrogen bonding with liquid water. To satisfy their hydrogen bonding requirement, water molecules that surround a hydrophobic compound order themselves by hydrogen bonding with each other, and consequently, lose many degrees of freedom. The reduction of exposed hydrophobic surfaces through protein folding; leads to a release of ordered layers of water molecules, and consequently, the entropy of the solvent increases. This increase in the entropy of the system is the basis for the hydrophobic effect, which leads to proteins adopting compact shapes. In terms of protein design, the essential property of water is the partitioning of polar and apolar residues between the protein surface and interior, or core. As a result of the hydrophobic effect, apolar residues are preferentially, but not always, buried in the protein interior, where the aqueous solvent is excluded. Conversely, polar residues may occasionally be buried but are preponderantly found on the protein surface; charged residues are rarely buried.

Due to the large number of water molecules in the layers surrounding the protein surface, water cannot be explicitly modeled in order to consider the effect of solvent on sequence preferences. Eisenberg and McLachlan (1986, Nature 319:199-203) showed that the free energy of interaction of a protein with water could be represented by the sum of the interaction energies of each atom of the protein with solvent. They further proposed that the interaction strength is proportional to the accessible surface area of each atom (ASAᵢ).

In a preferred embodiment of the present invention, water is modeled implicitly (in bulk, rather than as discrete molecules) and the solvation potential energy term is calculated from the difference in accessible surface area of each atom i in the folded and denatured protein (ΔASAᵢ) and from empirically determined solvation parameters for each atom (σᵢ), as shown in Equation V:

Eₛₒₗᵥ=Σ_{all atoms i}σᵢΔASAᵢ (V)

Accessible surface areas depend only on the atomic configuration of the protein and are calculated using the method of Lee and Richards (1971, J. Mol. Biol. 55:379-400) and the numerical surface calculation (NSC) routine of Eisenhaber et aL (1995, J. Comp. Chem. 16:273-284). A water molecule with radius of 1.4 Å is the “probe” that is rolled along the van der Waals surface of the protein atoms in order to calculate the accessible surface. The atomic radii and solvation parameters used to calculate the accessible surface areas of proteins in a preferred embodiment of the present invention are listed in Table 1. Correct accessible surface area measurements can only be made in the context of a full structure, and not before the optimal combination of side chain rotamers is found for the evaluated sequence.

TABLE 1
Atomic Solvation Parameters

| | | Radii | Solvation Parameters |
|---|---|---|---|
| | Atoms | (Å) | (cal/Å²) |
| Hydrophobic Atoms | C | 1.9 | 16 |
| | S | 1.8 | 21 |
| Polar Atoms | N | 1.7 | −6 |
| | O | 1.4 | −6 |
| Charged Atoms | N⁺ | 1.7 | −50 |
| | O⁻ | 1.4 | −24 |

In order to evaluate the generic scoring function (I) for different sequence variants and conformations during optimization, changes in ASA values for pairs of residues upon folding should be determined. However, this leads to an overestimation of the area of buried surfaces. Thus, in a preferred embodiment of the present invention, polar and apolar buried surfaces evaluated in a pairwise manner are scaled down in the manner proposed by Street and Mayo (1998, Folding & Design 3:253-258) in order to include a salvation parameter during optimization routines. The surface area of residue i buried by the template is evaluated as the difference between the accessible surface area of the same residue placed at the center of a five residue peptide and the accessible surface area of the residue in the target

$${BSA}_{i} = {ASA}_{i}^{5 \text{-} peptide} - {ASA}_{i}^{\begin{matrix}target \\ structure\end{matrix}} \qquad ( VI )$$

The surface area buried between residues i and j is evaluated as the difference between the exposed surface area of each residue separately placed in the target conformation and the exposed surface area of the pair of residues placed together in the target protein conformation. This is shown as follows:

$${BSA}_{ij} = λ ( {ASA}_{i}^{\begin{matrix}target \\ structure\end{matrix}} + {ASA}_{j}^{\begin{matrix}target \\ structure\end{matrix}} - {ASA}_{i and j}^{\begin{matrix}target \\ structure\end{matrix}} ) \qquad ( VII )$$

To compensate for the overestimation of total buried surface area, the ASA terms are scaled with a factor, λ, that depends on the location of residues i and j. In one embodiment, λ is taken to be 0.40 for core residues, 0.75 for non-core residues, and 0.60 for a pair that consists of one core residue and one non-core residue. In a preferred embodiment, % can be related to an alternative, pre-calculated parameter, Λ:

$$λ = Λ \frac{{N}_{first rotamer}^{contacts} + {N}_{second rotamer}^{contacts}}{{N}_{first rotamer}^{contacts} {N}_{second rotamer}^{contacts}}$$

The solvation energy (Equation V) is obtained by summing equations VI and VII over all side chains and by multiplying the accessible surface areas by the solvation parameters 0.100 for polar buried surfaces and −0.026 for nonpolar buried surfaces.

### 5.5.2. ENTROPY OF THE MAIN CHAIN

The entropy change upon folding, another major component of protein stability, is calculated in a preferred embodiment of the present invention using a statistical approach. Although entropy is a unified physical concept, it is practical to divide the entropy change into parts related to either the main chain or to the side chains (see Section 5.9, below). The main chain entropy term is expressed as the cost to fix the backbone conformation into the ensemble of φ and ψ dihedral angles of the target structure. These costs depend on the nature of the amino acid located at each φ-ψ pair, and were predetermined for use in the preferred embodiment of the invention to reflect the secondary structure propensities of the twenty amino acids (Muñoz & Serrano, 1994, Proteins 20(4):301-31 1), as described below.

A set of 527 protein structures that share less than 35% sequence homology (PDBSELECT; Hobohm & Sander, 1994, Protein Sci. 3:522-524; Hobohm et al., 1992, Protein Sci. 1:409-417) was used to obtain all main chain dihedral angles. The numbers of occurrences of each amino acid in regions of the Ramachandran (φ-ψ) plot sampled at fixed intervals, d₀ were determined. In a preferred embodiment, d₀ is taken to be twenty degrees. The tendency for amino acid X to populate a particular region of Ramachandran space, e.g., φₗ-ψᵢ, is the ratio of the number of hits in the interval considered (Nφᵢ-ψₗ) and the total number of hits for amino acid X (N all φ-ψ):

P^{X}_{φi-ψi}N^{X}_{φl-ψl}/N^{X}_{all φ-ψ} (VIII)

For such a partitioning of dihedral angle space, it is also necessary to quantify the distance, d, between pairs of points (each of which represents an residue conformation):

d_{φψ20°×20°}=\sqrt{(φᵢ−φ_{20°×20°})²+(ψᵢ−ψ_{20°×20°})²}

For 20×20 degree regions which are more than a threshold distance (in angle space), dₜ, from the residue φᵢ−ψᵢ dihedral angles, the occurrences are modified with a weight given by an exponential decay function of the separation distance, (to the inventors' knowledge, such an approach has not been used before in computer-based protein design methods):

ω_{φψ20°×20 °}=exp(−d_{φψ20°×20°}/d₀)

This modification allows a smooth transition of the energy function over the main chain dihedral angle space (instead of the abrupt changes that occurred previously when crossing the 20×20 boundaries). Thus, empirical observation is used to calculate the likelihood that a particular amino acid will have main chain dihedral angles φᵢ and ψₗ in any given protein structure.

In the preferred embodiment, the database used is large enough to be considered as a system under thermodynamic equilibrium in which pseudo-energies or costs to displace the equilibrium toward a particular state can be calculated from the natural logarithm of the ratio in Equation VIII. Entropy costs to fix the main chain dihedral angles of amino acid X in a particular region of the Ramachandran plot, e.g., φᵢ-ψᵢ, were therefore calculated as shown in the following equation for use in a preferred embodiment of the present invention:

$$- {RT}_{phys} ln \frac{{\sum }_{\begin{matrix}subspaces {φψ}_{20 ° \times 20 °} \\ close to φψ\end{matrix}} {w}_{{φψ}_{20 ° \times 20 °}} {N}_{{φψ}_{20 ° \times 20 °}}^{\begin{matrix}residue \\ type\end{matrix}}}{{N}_{all φψ}^{\begin{matrix}residue \\ type\end{matrix}}}$$

### 5.6. CONSIDERATION OF THE DENATURED STATE OF PROTEINS

An important consideration when modelling proteins is that of a reference state or configuration. In practice, proteins in solutions are dynamic ensembles of structures: the folded (“native”) structures are in equilibrium with unfolded “denatured” configurations. The former are compact, with residues in close contact with non-neighbouring residues due to the intricacies of the backbone configuration, whereas the latter are open, more chain-like. For the purposes of the present invention, the essential difference between these two extremes is that individual residues (particularly their side chains) participate in many more pairwise interactions amongst themselves in the folded state than in the unfolded. It is desired to quantify this difference at the level of individual residues, a result which is achievable as described below.

Whereas native protein structures are available (in the PDB) denatured structures must be obtained via simulation. In a preferred embodiment, a set of non-homologous proteins (obtained from the WHATIF database) is used to extract all protein fragments that are at least 4 and at most 20-residues long. These peptide segments may be clustered into groups according to length and structural homology, using a combination of main chain dihedral angle comparisons and internal (i.e. Cα-Cα) distance comparisons. There are many clustering algorithms which may be used for this purpose, for example, Ward's, Jarvis-Patrick and assorted hierarchical methods. For each cluster, a single representative is selected (for example, from the geometrical center of the cluster). The ensemble of representatives is used as a set of main chain templates to reconstruct sequences of the type (Ala)ₘ-X-(Ala)ₙ and (Ala)ₗ-X-(Ala)ₘ-Y-(Ala)ₙ where X and Y are any of the 20 natural amino acids (and the subscripts, l, m, n, represent segment lengths) and Ala represents the amino acid, alanine. (These sequences represent the amino acid residues of interest in an ordinary environment: alanine is the amino acid with the smallest (shortest) carbon containing side chain. It therefore contains no polar or bulky groups which might cause folding or twisting of the sequence but, unlike glycine (whose side chain is hydrogen), has sufficient bulk to prevent collapse.) Perla itself can be used to determine a solution score for each sequence, i.e., each peptide fragment. It is then possible to compute an average solution score that corresponds to the output of a partition function measured over the ensemble of fragments. Perla does so for each separate energy term, and then provides sets of values to be used as reference values for the random coil, i.e., the denatured state. The references for the intrinsic parts of the van der Waals, hydrogen bonding and electrostatic energy terms, and the side chain rotamer entropies, are measured with sequences of the type (Ala)ₘ-X-(Ala)ₗ, while the references for the pairwise parts of the van der Waals, hydrogen bonding and electrostatic energy terms, are measured with sequences of the type (Ala)ₗ-X-(Ala)ₘ-Y-(Ala)ₙ.

For application to other categories of macromolecules, it may be appropriate to consider an alternative form of reference state. For example, when attempting to find a set of nucleotides that improves the interaction between a nucleic acid and a protein or other nucleic acid sequence, the reference may be the isolated nucleic acid in question, whereas the scoring function will quantify the extent of the interaction.

### 5.7. CONSTRUCTION OF THE ROTAMER LIBRARY USED IN PERLA

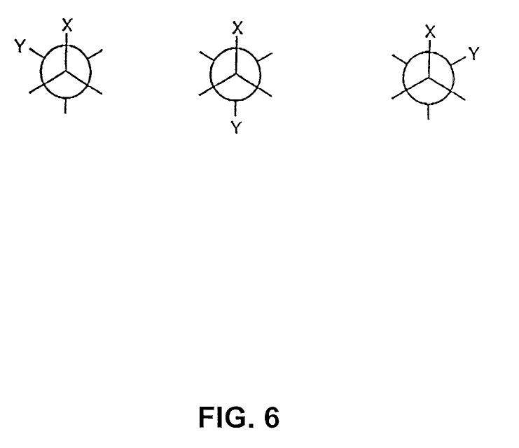

Crystallographically determined protein structures show that the side chain dihedral angles are not distributed uniformly through 360° (Janin & Wodak, 1978, J. Mol. Biol. 125:357-386; Ponder & Richards, 1987, J. Mol. Biol. 193:775-791). For example, the torsion angles around two bonded sp³ carbons generally cluster into ‘gauche+’ (+60°), ‘gauche−’ (−60°), and ‘trans’ (180°) conformations (FIG. 6). In a preferred embodiment of the present invention, we wish to construct rotamer libraries in which all significant conformers are representated.

By way of example, the set of 527 protein structures that share less than 35% sequence homology (see Section 5.5; PDBSELECT; Hobohm & Sander, 1994, Protein Sci. 3:522-524; Hobohm et al., 1992, Protein Sci. 1:409-417) was used to obtain all sets of side chain dihedral angles (_{102 1}, _{χ2}, _{102 3}, ψ₄) For each amino acid other than glycine and alanine, the distribution υ(χ) of dihedral angles determined from the Protein Data Bank structures was fitted to a combination of normal distributions represented by a sum of Gaussians, as follows:

$$υ ( χ ) = k + {\sum }_{i = 1}^{N} ( 1 / {σ}_{i} \sqrt{2 π} ) exp ( - {( χ - {μ}_{i} )}^{2} / 2 {σ}_{i}^{2 } ) \qquad ( IX )$$

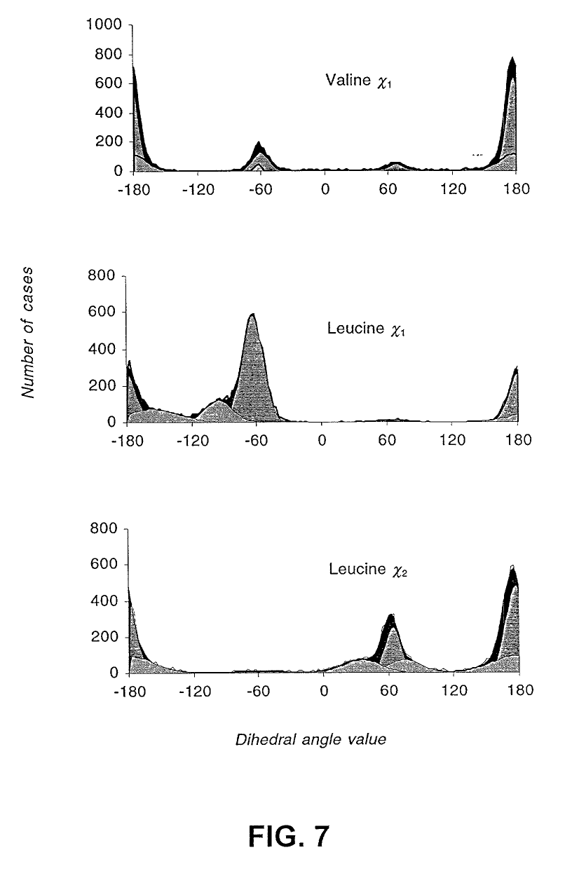

The number of Gaussian terms, N, was modified until no further reduction of the square of the difference between observed and calculated distributions was obtained. A constant term, k, was added to fit the distribution of side chain dihedral angles with poorly marked preferences, e.g., χ₂ of Asn and Asp, or to represent the noise. The outputs of the fitting procedure are the centers (μᵢ) of the N Gaussian peaks and their standard deviations, σᵢ. In addition to the expected well-defined peaks of standard conformers, separate distributions of lower amplitudes were used to fit the data. In most cases, as illustrated for valine (light grey peaks in the top panel of FIG. 7), the additional Gaussian curves represent variations around different dihedral angle values that can be adopted by a particular amino acid in a significant number of instances.

Side chain rotamers with all combinations of the peak centers, μᵢ (except for “ghost” peaks) were constructed using ideal values for the covalent bonds and angles (Mazur & Abagyan, 1989, J. Biomol. Struct. Dyn. 6(4):815-832; Momani et al., 1975, J. Phys. Chem. 79:2361-2381; Nemethy et al., 1983, J. Phys. Chem. 87:1883-1887). For dihedral angles that do not have a normal distribution, e.g., χ₂ of asparagine and aspartic acid, and χ₃ of glutamine and glutamic acid, sets of values were chosen to sample the range of observed values. The constructed side chains were inspected visually and conformations with steric clashes were removed. The remaining side chain conformations form the custom-made rotamer library of 419 rotamers employed by the preferred embodiment of the present invention, (Table 2). Side chains built by the preferred embodiment of the present invention are taken from this library. The advantage of this approach for the construction of a rotamer library is that it does not use stereochemical rules to generate the rotamers, thus allowing for the addition to the library of less abundant but relevant rotamers. Furthermore, the fitted normal distributions define the margins within which the rotamers can oscillate during evaluation of sequences.

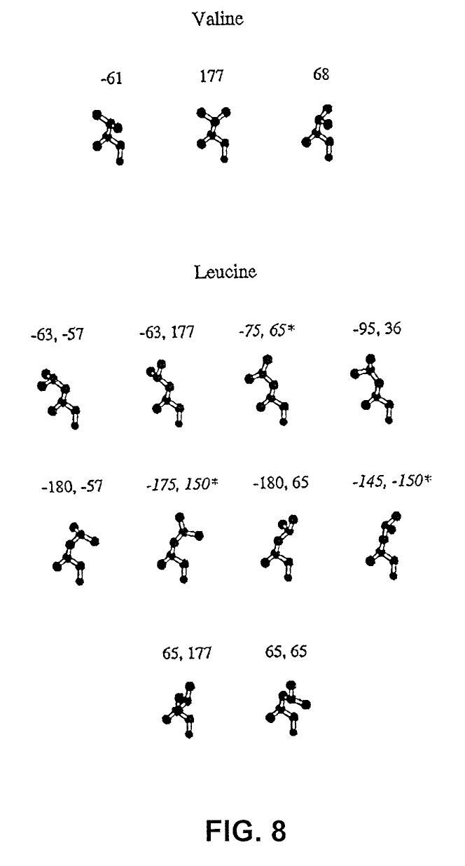

The generation of dihedral angles by means of Equation IX does not include the fact that consecutive angles have correlated distributions. The correlation, due to the topological structures of certain amino acids, can be so strong that a particular value of χ₁ is only possible if χ₂ itself adopts a defined value (e.g., the −95, 36 conformer of leucine in FIG. 8). Furthermore, the fitting procedure used to generate the dihedral angles for the rotamers used in the library cannot detect rare peaks.

More preferably, side chain conformations with correlated dihedral angles or rare dihedral angles can be included in the rotamer library by repeating the analysis above while considering the distribution of all dihedral angles of an amino acid simultaneously, since rare peaks are more resolved in a multidimensional representation. The rotamer library employed by the methods of the present invention contains only a few identified cases of rare or correlated dihedral angle values, e.g., the −175, 150 and −145, −150 leucine conformers, (FIG. 8).

The calculation of side chain entropy terms is described subsequently in the discussion of the mean field approximation.

TABLE 2
Summarized Description of Rotamer Library

| | Dihedral | Number of | | Dihedral | Number of |
|---|---|---|---|---|---|
| Amino Acid | Angles | Rotamers | Amino Acid | Angles | Rotamers |
| Ala | — | 1 | Met | X₁, X₂, X₃ | 27 |
| Cys | X₁, X₂ | 18 | Asn | X₁, X₂ | 18 |
| Asp | X₁, X₂ | 9 | Pro | X₁, X₂, X₃ | 3 |
| Glu | X₁, X₂, X₃ | 27 | Gln | X₁, X₂, X₃ | 54 |
| Phe | X₁, X₂ | 9 | Arg | X₁, X₂, X₃, X₄ | 75 |
| Gly | — | 1 | Ser | X₁, X₂ | 18 |
| His | X₁, X₂ | 12 | Thr | X₁, X₂ | 18 |
| Ile | X₁, X₂ | 9 | Val | X₁ | 3 |
| Lys | X₁, X₂, X₃, X₄ | 77 | Trp | X₁, X₂ | 12 |
| Leu | X₁, X₂ | 10 | Tyr | X₁, X₂ | 18 |

TOTAL: 419

In addition, a new energy term is used that can be understood as a rotamer vibration entropy , that goes into the intrinsic and pairwise energy terms as follows: For the intrinsic term it is:

$$- T ( - R {\sum }_{sub \text{-} rotamers i} {w}_{i} ln {w}_{i} + R {\sum }_{sub \text{-} rotamers} {N}_{sub \text{-} rotamers}^{- 1} ln {N}_{sub \text{-} rotamers}^{- 1} )$$

For the pairwise term, it is:

$$- T λ ( \begin{matrix}- R {\sum }_{\begin{matrix}pairs of \\ sub \text{-} rotamers ij\end{matrix}} {w}_{ij} ln {w}_{ij} + \\ R {\sum }_{\begin{matrix}sub \text{-} rotamers i \\ first side chain\end{matrix}} {w}_{i}^{first side chain} ln {w}_{i}^{first side chain} + \\ R {\sum }_{\begin{matrix}sub \text{-} rotamers j \\ second side chain\end{matrix}} {w}_{j}^{second side chain} ln {w}_{j}^{second side chain}\end{matrix} )$$

The intrinsic vibrational entropy term represents the change from a uniform distribution to the distribution obtained from the partition function described as the equation of Section 5.8.2, and the pairwise vibration entropy term is the change from the initial and main chain-dependent distributions of sub-rotamers of the two side chain rotamers to the distribution of sub-rotamer pairs due to their interaction with each other. Scaling by a factor λ is necessary to avoid the overestimation of the entropy change when summing over all ²⁰pairs of interacting rotamers. In the preferred embodiment of the present invention, λ is:

$$λ = Λ \frac{{N}_{first rotamer}^{contacts} + {N}_{second rotamer}^{contacts}}{{N}_{first rotamer}^{contacts} {N}_{second rotamer}^{contacts}}$$

Here, Λ is set to be 0.5.

### 5.8. ENERGY MINIMIZATION AND ELIMINATION OF INCOMPATIBLE AMINO ACID CONFORMERS

There are two facets of the energy minimization: minimization of the interaction between side chain and template; and minimization of the pairwise interactions between side chains. In either case, there are at least two possible methods of minimization.

### 5.8.1. METHODS OF ENERGY MINIMIZATION

Side chain conformations taken from the rotamer library of the preferred embodiment of the present invention are idealized. Consequently, in the preferred embodiment of the present invention, their interactions with the protein template and other side chains are optimized and flexibility is introduced to relax strain inherent in the fixed geometries provided by the rotamer library.

Energy minimization is carried out in dihedral angle space using the non-bonding terms of the molecular mechanics force field (van der Waals, electrostatic, and hydrogen bonding).

In one embodiment of the present invention, the energy minimization method may be so-called “Steepest descent”; in another, it may be taken from a class of methods known as “quasi-Newtonian”. The theory behind these methods is accessible to one skilled in the art (and can be found in Numerical Recipes in C—The Art of Scientific Computing by WH Press, 2^{nd} edition section 10.6, S A Teukolsky, W T Vetterling and B P Flannery), but in general terms, the interaction energy and its gradient with respect to displacements in dihedral angle space are utilized. Minima on the energy surface are located iteratively through use of the gradient to search downhill from a given point. The quasi-Newton methods attempt to gather information about the curvature of the energy surface. Methods in this category include “BFGS” and “conjugate gradient”, the distinctions between them arising in how each decides to approximate the Hessian (matrix of second derivatives of the energy). In practice, the method of conjugate-gradient has been found to be effective, provided that certain precautionary measures are taken in order to avoid large rotations that would in fact transform one side chain rotamer into another one (this would deteriorate all subsequent partition functions, i.e., that calculated to eliminate the rotamers that have the lowest probabilities according to the interaction energy with the main chain). First, the rotation step size has to be small (fractions of a degree to a few degrees); thus the factors that multiply the gradients are small, and the gradients themselves are capped at some energy maximum. Second, the energy function is modified to contain a rotation penalty function to directly limit the minimization sampling to conformations close to the initial rotamer structure; it has the following form:

$${\sum }_{minimized x} {{k}_{x} ( x - {x}_{o} )}^{2}$$

The “force constants” kₓ are expressed as functions of the standard deviations measured during the construction of the rotamer library by fitting of the frequency distributions observed in the protein database. Third, if a large rotation (more than a couple of standard deviations) is done despite the penalty, the rotamer is simply placed back in its initial conformation, discarding the result of the minimization.

In a preferred embodiment of the present invention, minimization is carried out by exhaustive sampling of dihedral angles of rotamers close to the ideal conformation. This method is not only simpler, but is superior to conjugate-gradient and similar methods of optimization. (As an example of why this is so, the formation of a hydrogen bond may require an energetically unfavorable rotation of a side chain in order for the correct geometry to be achieved, which would only be discovered using a method that samples rotamer conformations close to the ideal conformation without energy minimization.) The conformations which are obtained through systematic rotations around the dihedral angles χ₁ and χ₂ are referred to as “sub-rotamers”. In a preferred embodiment of the present invention, the step size and number of steps are precomputed for each of the twenty naturally occurring amino acids and are determined to cover rotations smaller than two standard deviations away from the minima in dihedral angle space (derived during the creation of the rotamer library). Such a range is usually about 15 degrees, or even smaller than a single standard deviation if it is necessary to optimize the residue set. It is expected that a sequence which enables the packing of rotamers in their ideal conformation (that of the library) should be preferred to another sequence that would necessitate rotations of its side chain rotamers. Although there is an advantage in using many small steps in the thoroughness of coverage, the calculation time has to be considered. In the preferred embodiment, three steps of 5 degrees in each direction around the dihedral angles give good results, i.e., 7 steps in all for each angle. This leads to 49 sub-rotamers, and 49×49 energy calculations to obtain a pairwise energy term.

### 5.8.2. MINIMIZING INTERACTION OF SIDE CHAIN AND TEMPLATE

When considering the interaction of the side chain with the template, the final energy is the weighted average over all possible sub-rotamers. The partition function that defines the weight of state i as a part of a system with N states with energies Eᵢ is defined as follows:

$${w}_{i} = exp ( - {E}_{i} / RT ) / {\sum }_{j ( including i )}^{N} exp ( - {E}_{j} / RT )$$

### 3.3. MINIMIZING INTERACTIONS BETWEEN PAIRS OF SIDE CHAINS

In a preferred embodiment of the present invention, pairs of side chains are minimized using systematic sampling of sub-rotamers. The outcome of using sub-rotamers to optimize pairs of side chains is the weighted average of all possible pairs. Minimizing pairs of side chains individually (regardless of other side chains) is assumed to be correct as long as the actual conformations of the side chains depart only slightly from their starting point (to prevent any incompatibility in larger sets of side chains).

### 5.8.4. PROCESSING RESULTS OF MINIMIZATION

In the method of the present invention, side chain conformers that do not interact favorably with the protein target conformation after minimization are rejected. In one embodiment of the present invention, rotamers for which the intrinsic energy term is above a predetermined threshold are rejected. The use of an absolute threshold, however, may not be ideal. Intrinsic energies vary in magnitude from site to site because they depend on the actual backbone structure. Poorly resolved structures tend to show many spots with local repulsions. Moreover, strains do exist in proteins where repulsions are common, e.g., in turns, where the tight reversal of the main chain in a turn is often accomplished by placing the φ-ψ dihedral angles in less favored regions of the Ramachandran plot. Therefore, in a preferred embodiment of the present invention, the absolute energy threshold is placed high enough (about 50 kcal/mol) to accept enough side chain conformers at every position of the target structure, and a relative threshold is then applied to keep only the most qualified rotamers. The relative threshold is determined by calculating for each amino acid a partition function over all rotamers using the template-side chain interaction terms, with the minimum threshold being fixed as a minimal probability of frequency, e.g., 0.001.

The way in which minimization may be applied to other categories of macromolecules will depend upon the complexity of the building blocks and upon the overall structure of the macromolecule. In long extended systems such as nucleic acids, the relevance of the pairwise energy term will be less important. Similarly, in systems with inflexible sidechains or with little conformational flexibility, the need for a thorough minimization protocol will be diminished.

### 5.9. OPTIMIZATION ROUTINES

For peptides longer than a few residues, an exhaustive sampling of every possible sequence and combination of rotamers is not practical. Hence, in the methods of the present invention, procedures are used which either decrease the size of the sequence space to be covered or rapidly find the probabilities of having particular rotamers at each position of the modeled protein structure. These procedures are often termed “semi-exhaustive”.

### 5.9.1. DEAD-END ELIMINATION

In the method of the present invention, significant reductions of sequence space are obtained by discarding amino acids that cannot belong to the optimal sequence, which is that with the lowest potential energy, and the calculation time is shortened. To eliminate an amino acid, the modeling procedure has simply to exclude all its known side chain conformations. Undesired rotamers are detected by applying the dead-end criterion, which is the underlying principle of the dead-end elimination (“DEE”) theorem. The theorem states that, for a given residue i, a particular rotamer, iₗ, is not compatible with the global minimum energy conformation (“GMEC”) if, for the same residue i, an alternative rotamer iₜ exists for which the following inequality holds true (Desmet et al., 1992, Nature 356:539-542):

$${E}_{{i}_{r}}^{template} + {\sum }_{j \neq i} {min}_{s} ( {E}_{{i}_{r} {j}_{s}}^{pairwise} ) > {E}_{{i}_{t}}^{template} + {\sum }_{j \neq i} {max}_{s} ( {E}_{{i}_{t} {j}_{s}}^{pairwise} ) \qquad ( X )$$

The minimum and maximum functions (min, and max.) cycle over all rotamers jₛ of residues j, searching for the rotamer which offers, respectively, the lowest and highest value of the interaction energy with residue i. The rotamers picked by the minimum or maximum function, jₛ, do not have to be identical. Thus, the left-hand side of Equation X evaluates the best possible interaction of rotamer i, with the side chains of all other modeled residues, while the right-hand side evaluates the worst possible interaction of an alternative rotamer iₜ for the same residue with all the other modeled residues. A side chain rotamer is “dead-ending” if its best interaction with the surroundings is less advantageous than that for another rotamer of the same side chain taken at its worst. Only one rotamer iₜ that satisfies the inequality has to be found to qualify iᵣ as dead-ending.

In another embodiment of the invention, a more powerful version of the elimination criterion is utilized that is less restrictive and therefore more effective (Goldstein, 1994, Biophys. J. 66:1335-1340). It states that a side chain rotamer iᵣ is dead-ending if the energy of the model can be lowered by the choice of an alternative rotamer iₜ, while keeping all other side chains fixed. This elimination criterion is described in Equation XI for the same set of jₛ:

$${E}_{{i}_{r}}^{template} - {E}_{{i}_{r}}^{template} + {\sum }_{j \neq i} {min}_{s} ( {E}_{{i}_{r} - {j}_{s}}^{pairwise} - {E}_{{i}_{r} {j}_{s}}^{pairwise} ) > 0 \qquad ( XI )$$

Both dead-end elimination criteria can be extended to pairs of rotamers. The energy contribution of a rotamer pair (iᵣ,jₛ) and its interaction with a third residue/rotamer kₜ are given by Equations XII and XIII, respectively:

$${E}_{( {i}_{r} - {j}_{s} )} = {E}_{{i}_{r}}^{template} + {E}_{{j}_{s}}^{template} + {E}_{{i}_{r} {j}_{s}}^{pairwise} \qquad ( XII )$$

E₍ᵢ_{ᵣ}₋ⱼ_{ₛ}₎ₖ_{ₛ}=E₍ᵢ_{ᵣ}₋ₖ_{ₗ}₎+E₍ⱼ_{ₛ}₋ₖ_{ₗ}_{) (}i≠j≠k) (XIII)

Then, Equation X, in one embodiment of the present invention, can be written for pairs of rotamers, as follows:

$${E}_{( {i}_{r} , {j}_{s} )} + {\sum }_{k \neq i , j} {min}_{i} ( {E}_{( {l}_{r} , {j}_{s} ) , {k}_{l}} ) > {E}_{( {i}_{u} , {j}_{v} )} + {\sum }_{k \neq i , j} {max}_{i} ( {E}_{( {i}_{u} , {j}_{v} ) , {k}_{l}} )$$

and in a preferred embodiment of the present invention, Equation XI, can be rewritten for pairs of rotamers as follows:

$${E}_{( {i}_{r} , {j}_{s} )} - {E}_{( {l}_{u} , {j}_{v} )} + {\sum }_{k \neq i , j} {min}_{i} ( {E}_{( {i}_{r} , {j}_{s} ) , {k}_{i}} - {E}_{( {i}_{u} , {j}_{v} ) , {k}_{t}} ) > 0 \qquad \text{(XIV)}$$

Dead-ending pairs do not lead to an elimination of a particular amino acid unless one of the participating rotamers is the only possible side chain conformer for the related residue position, in which case the other rotamer of the pair is not compatible with the GMEC and can be discarded. Lasters and co-workers (Lasters et al., 1995, Protein Eng. 8:815-822; Lasters and Desmet, 1993, Protein Eng. 6:717-722) showed that dead-ending pairs can be safely ignored in the minimum term of Equation X and the left-hand term of the minimum function of Equation XI. Due to the exclusion of dead-ending pairs, the minimum functions might return higher values that strengthen the rotamer elimination criterion.

In the preferred embodiment of the present invention, the dead-end elimination routine follows an iterative process as follows: (a) dead-ending rotamers are eliminated, repeating evaluations of Goldstein's criterion (Equation XI) until no more dead-ending rotamers are found; (b) dead-ending pairs are detected using the first elimination criterion (Equation XV; Desmet et al., 1992, Nature 356:539-542) because it is estimated more quickly; and (c) new cycles of rotamer removal as in step (a) are carried out. This continues until no more dead-ending pairs can be found. The more effective but computationally expensive criterion for the detection of dead-ending pairs (Equation XIV) is used when many rotamers have been eliminated and the whole cycle is restarted. At the end, if all rotamers of an amino acid at a particular site in the protein are dismissed, sequences containing this particular amino acid at that site are also abandoned.

In one embodiment of the present invention, the DEE routine can be used to determine an optimal set of rotamers for a given sequence. In a preferred embodiment, the routine is not used to limit the output to one optimal set of rotamers, since side chains, particularly those positioned on the solvent-exposed surface, are flexible and adopt different configurations.

### 5.9.2. MEAN FIELD THEORY

The foregoing will have provided the user with one or more acceptable rotamers for each residue position. This outcome represents a level of statistical uncertainty in the situation being described. It is not adequate to subsequently model the system with merely one rotamer for each residue.

It is desirable to obtain a contribution to the entropic term from all possible conformations of side chains that remain after iterative dead-end elimination has eliminated all sequences that cannot belong to the global energy minimum, as described above in this section. In a preferred embodiment of the present invention, mean field theory (MFT) is utilized to achieve this. MFT is an iterative technique which has found wide application in the physical sciences for describing systems of interacting particles which may adopt many different energy states.

All possible side chain conformations are considered using a mean field approximation that is designed to provide an estimate of the entropy of side chains in both the denatured and the modeled state, i.e., the template. The method attributes to each side chain conformation of all residues in the protein sequence that are not fixed a probability that depends on the average of all possible environments, weighted in turn by their respective probabilities of occurrence (Koehl & Delarue, 1994, J. Mol. Biol. 239:249-275; Koehl & Delarue, 1995, Nat. Struct. Biol. 2:163-170; Koehl & Delarue, 1996, Curr. Opin. Struct. Biol. 6:222-226). The probability of occurrence is related to the energetic favourability. The system is initialized by giving an equal probability to each side chain rotamer so that every side chain conformation “feels” equally the presence of all rotamers at other residue positions. At this point, the field energy of each rotamer is the sum of all possible pairwise interaction energies normalized by the number of interactions, plus a contribution from the interaction with the protein template, as shown as follows:

$${MF}_{{i}_{r}} = {E}_{{i}_{r}}^{template} + {\sum }_{\begin{matrix}all residues \\ j\end{matrix}} {\sum }_{ {all rotamers}_{s}} {w}_{{j}_{s}} {E}_{{i}_{r} {j}_{s}}^{pairwise}$$

wherein MF(iᵣ) is the mean field energy experienced by rotamer r of residue i. Initially, the probabilities, ω, for the rotamers of any residue are equal. At all times, in order for them to be interpretable as such, these probabilities sum up to 1. The application of MFT is to minimize the term MF by suitable adjustments of the weights.

Having obtained the mean field energy perceived by each rotamer of a residue, proper weights can be estimated from a partition function that integrates all field energies, so that the rotamer that interacts best with its environments becomes more probable than competing rotamers:

$${w}_{{i}_{r}} = \frac{exp ( \frac{- {MF}_{{i}_{r}}}{RT} )}{{\sum }_{\begin{matrix}all rotamers l \\ of residue i\end{matrix}}^{N} exp ( \frac{- {MF}_{{i}_{t}}}{RT} )} \qquad ( XV )$$

Thus, equation XV for the weight of a particular rotamer is the partition function that defines the probability of having the rotamer iᵣ, from a system of N rotamers. R and T are the gas constant and the temperature, respectively.

Several iterations of field energy calculations (now the weighted average) and partitioning are conducted until the set of probabilities is not further modified. It should be clear that this process is “non-linear”, i.e., the quantity to be minimized, MF, depends on itself through the adjustable quantities, ω. In such circumstances, convergence may be achieved through one of several methods of non-linear optimization that will be familiar to one skilled in the art. In a preferred embodiment of the present invention, it has been found that smooth progress toward the equilibrium state of convergence, is effectively achieved via “simulated annealing”. This technique is a computer-based method, which simulates the “heating” of the protein structure to a high temperature followed by “cooling” it. This is done because the high starting temperatures of simulated annealing, e.g., 1000 K, lead to monotonic distributions of probabilities of side chain conformations, thus providing a random and unbiased starting sample of rotamers. The system is then cooled down to sharpen the distributions and optimize the sets of side chain conformations.

The advantage of the mean field method is that the estimated probabilities are similar to frequencies of occurrence, and are coupled to entropy, as shown in the following Equation:

$${S}_{i} = - R {\sum }_{\begin{matrix}all rotamers \\ t\end{matrix}} {w}_{{i}_{t}} ln {w}_{{i}_{t}}$$

wherein Sᵢ is the side chain conformational entropy of residue i, R is the gas constant, and the set of ω represent the probabilities of occurrence of each rotamer.

In a preferred embodiment of the present invention, the mean field approximation is used to estimate the weights of all rotamers of the different amino acids in a set of protein fragments obtained from protein structures in the PDB. In another embodiment, amino acids embedded in sample 5-residue extended peptides are employed. From data obtained in this way, the reference entropy of the denatured state can be measured.

The change in entropy of side chains upon protein folding, in both rotamer and sub-rotamer space can therefore be obtained by a comparison of the entropy of the side chain in the native protein, which is then added to the previously determined entropy arising from the fixing of the protein backbone (See Section 5.5) to obtain the total change in entropy upon folding of the protein.

Mean Field Theory is particularly apposite for the study of amino acid sidechains in proteins. Alternative schemes may be employed both for the study of proteins and for applications to other macromolecules. In another embodiment, the iterative scheme used is Monte Carlo sampling.

### 5.10. RE-EVALUATION OF SOLVATION ENERGIES FOR SEQUENCES WITH LOW SCORING FUNCTIONS

In the preferred embodiment of the present invention, if the scoring function of a sequence, after being stripped of its solvation term, is below a predetermined energy threshold, solvation energies for that sequence are re-evaluated according to two criteria. Otherwise, the sequence is dropped from consideration. Solvation energies obtained in a pairwise manner are removed and re-computed from accessible surface areas derived from the optimized configuration of side chains. In the preferred embodiment, the more detailed solvation parameters of Eisenberg and McLachlan (1986, Nature 319:199-203, Table 1) may be used, though other parameter sets would be adequate. The accessible surface areas may be measured using the NSC routine (Eisenhaber et al. 1995, J. Comp. Chem. 16:273-284) or another equivalent method. The result is a more accurate calculation of the potential energy of the mutant protein.

The solvation energy is assessed according to two properties. As with the previous calculation of the energy, the solvation energy of the reference (denatured) state of the protein must be considered. This can be calculated for each amino acid by considering the solvation energy of a reference state. For this purpose, a reference can be obtained from the average solvent-exposure of the amino acid in 5-residue peptide sequence, as observed in the Protein Data Bank, but without the context of the surrounding protein structure (except for the “capping” residues on the N- and C- ends of the sequence. Each residue then behaves as if the sequence were a free chain. It is also necessary to consider the environment of the residue in the protein. For example, exposed hydrophobic residues should be penalised because they may lead to misfolding. Consequently, the solvation energy is calculated by comparing with residues in similar structures in the PDB. By doing this, it is possible to arrive at optimized conformations and sequences for protein-like solvation.

### 5.11. OUTPUT OF OPTIMAL SEQUENCE RESULTS

After the dead-end elimination procedure of the preferred embodiment of the present invention, many sequences remain; nevertheless, the subsequent steps (see Mean Field Theory and Refinement, above) ensure that only those conformations and sequences that satisfy predetermined energy thresholds finally surface as candidates for the target structure. The preferred embodiment of the present invention can produce either detailed or limited outputs, depending on the size of the sampled sequence space. In one embodiment, the output is a simple list of sequences and scores (evaluated using the scoring function) that can be sorted according to the calculated potential energy so that the lowest energy sequences may be readily identified. In another embodiment, a more complete output presents a numerical profile of the energy for each sequence produced. The program is also capable of producing a coordinate file (in PDB format) with the structure of the protein having a mutated sequence. If mean field sampling is performed, both the PDB-file and detailed energy outputs correspond to the combination of most probable rotamers. In another embodiment, the detailed energy output includes the effective solution score taking into account all candidate rotamers with the weights they were given, and a second detailed description of the separate pairwise energy terms resulting of the combination of all possible side chain rotamers. If DEE is used for the conformational sampling (without subsequent application of MFT afterwards), then the effective solution score corresponds to the GMEC where one and only one rotamer is retained for each amino acid side chain; the detailed energy file offers nothing else than the separate energy terms which produce the GMEC total energy and the PDB coordinate file represents the GMEC model.

### 5.12. GENERALISATION TO NON-PEPTIDES

Whilst the foregoing has focused specifically upon the types of macromolecules known as proteins and their building blocks, amino acids, the methods can readily be applied by one skilled in the art to other categories of macromolecules which can be viewed as comprising a fixed structure attached to which are freely rotating groups. The alternative embodiments which would be required concern the nature of the rotamer library, the choice of reference state, and the property of interest to optimise. Even though amino acid residues have a simplifying feature in that they consist of side chain and a fixed backbone which enables their conformations to be simply expressed as rotamer libraries, other building blocks may also be conveniently modelled by one skilled in the art. Sugar molecules and nucleotide bases have freely rotating groups attached to ring systems, simplifying structural features which would permit the straightforward construction of conformer libaries. Conformers can be obtained from known structures of carbohydrates and nucleic acids respectively or modelled computationally.

The idea of a reference state, although usefully expressed as the denatured form when modelling proteins, can be defined differently for other molecules. By analogy with the alanine pentapeptide, a reference saccharide molecule or small sequence of nucleotide bases could be established as a reference structure for carbohydrate or nucleic acid modelling, respectively, in a manner similar to the procedure already described. In other applications it may be useful to utilise an unsolvated molecule as the reference.

Solubility itself may be a property that can be the subject of investigation and optimization with Perla.

In applications where the property of interest is an interaction between the target molecule and some binding molecule, the reference state can be considered to be the unbound target molecule or a sum of contributions from the unbound target molecule and unbound binding molecule. This type of application is likely to be widespread, for example: the interaction between DNA and a protein (e.g., a transcription factor); RNA and a protein; the interaction between peptides in solution; the interaction of a polar macromolecule and a lipophilic membrane.

### 5.13. A SYSTEM FOR OPTIMIZING STRUCTURAL UNITS OF A MACROMOLECULE

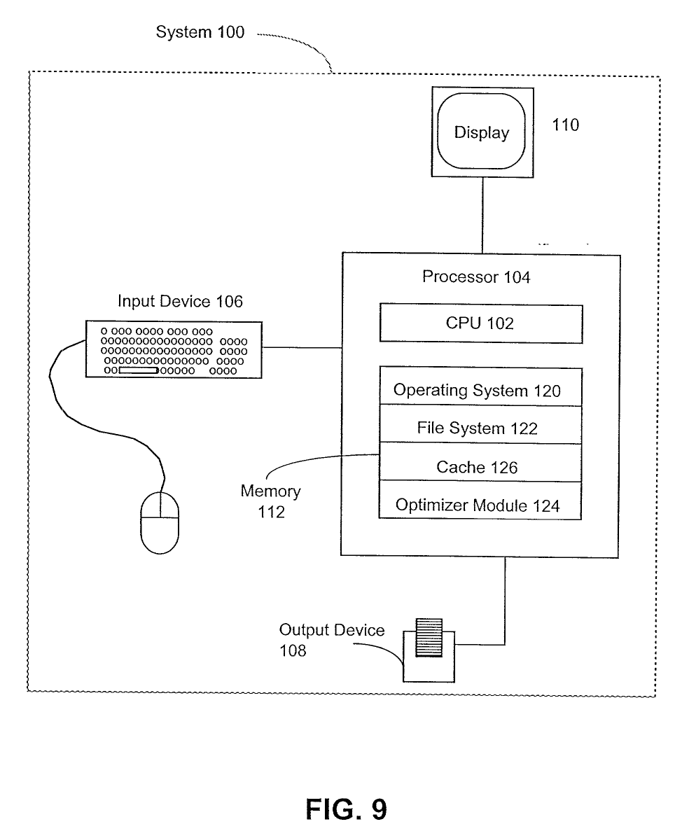

The present invention addresses the ability to engineer derivatives of large molecules through systematic variations of their structural components by presenting scores of preferred solutions after rigorous analysis of a user-defined search space. The invention, as shown in FIG. 9, comprises a system 100 for optimizing a set of structural units in a target macromolecule, comprising a processor 104 which consists of a central processing unit 102, an input device 106 for inputting requests, an output device 108, a section of main memory 112, and at least one bus connecting the central processing unit, the memory, the input device, and the output device. The memory stores an operating system, 120, a file system, 122, cache 126 and an optimizer module 124 configured to optimize a set of structural units in the target macromolecule. The macromolecule, which may be a peptide, protein, strand of DNA or RNA or a carbohydrate, or any organic molecule which consists of identifiable distinct structural units, has a 3D structure whose geometry is known to atomic resolution. The optimizer module, upon receiving a request to optimize the set of structural units and at least one candidate building block for each unit in the set, utilises, for each candidate building block, one or more candidate conformations. For each determined candidate conformation, the optimizer substitutes a structural unit of the target macromolecule with the candidate conformation and calculates an intrinsic energy term of the candidate conformer. The optimizer module subsequently rejects candidate conformations having an intrinsic energy above a threshold value and calculates a pairwise interaction energy term for all possible conformations that have not been rejected by the threshold energy value criterion. The method enables determination of solutions, including a best solution corresponding to a global minimum energy conformation, which are ranked by solution score. Each building block in the set to be optimized is represented in each solution by one or more candidate conformations that were not rejected by the threshold energy criterion when substituted into the structure of the target macromolecule. Each solution score represents a difference between the summed potential energy of each candidate conformation substituted into the target structure and the same conformation substituted into a representation of its local environment in the target. This procedure attempts to factor out long-range interactions which are present in the target. The solution score comprises molecular mechanics energy terms (van der Waals, hydrogen bonding and electrostatic) and terms corresponding to an empirical potential (entropy and salvation) along with a user-defined statistical term.

This system, when operated in a laboratory environment can provide an efficient and useful method of directing experimental efforts towards engineering sequence variations in a target macromolecule. Said system, being capable of quantifying the potency of a plurality of sequences and thereby selecting a small number which would be worthy of synthesis, can operate in tandem with experiment to optimize properties of interest of the target macromolecule.

### 6. EXAMPLE

Parts of the SH3 domain of α-spectrin, a small and globular protein domain with a β-sheet architecture, were re-designed with Perla. Nine residues in the buried core of the domain were replaced by different hydrophobic amino acids. Four residues that form a surface-exposed turn were similarly redesigned, allowing both polar and non-polar amino acids.

Choice of template: The three-dimensional architecture (template), residue numbering and wild-type (WT) sequence used in the design correspond to the structure presented by Musacchio et al. 1992, (pdb accession code 1shg; Musacchio, a., Noble, M., Pauptit, R., Wierenga, R. and Saraste, M. Crystal structure of a Src-homology 3 (SH3) domain. Nature, 359, 851-855.

Operating Parameters:

For the various energy terms, recommended weights were set at 1.0.

When calculating molecular mechanics energy terms, an interaction cutoff was employed. (It is well-established that pairwise interactions between atoms separated by 30 greater than a certain distance contribute negligibly.) Here 20 Å is found to be large enough to avoid any important truncation of the electrostatic interaction energy; van der Waals interactions are only taken into account for atom-atom distances smaller than 8 Å.

Modeling Sets

In the following examples, Perla takes different amino acid side chains from its rotamer library and assembles them on top of nine buried, or four exposed, positions of the SH3 domain. The first modeling set (called CORE) consists of V9, A11, V23, M25, L31, L33, V44, V53 and V58. The second set (called SURFACE) comprises V46, N47, D48 and R49. Since all other side chains are kept in the conformation deposited at the protein data bank, they constitute the protein template, along with the main chain of the whole protein.

Amino Acids Considered

For the CORE modeling set, only nonpolar residues (AVILFW) were considered to speed up the sequence sampling, through reducing the total number of sequences. Polar and charged amino acids could be avoided since all residues were to be fully buried. Since there were 9 residues to design, the total number of sequences was 10⁷.

For the SURFACE modeling set, both polar and nonpolar amino acids were considered (AVILGDNSTEOKRYW); total number of sequences 10^{4,7}.

Numbers of Rotamers

For the CORE set, the amino acid considered have 1, 3, 9, 10, 9 and 12 rotamers, respectively, which means that 44 side chains are modeled onto the nine positions, resulting in 396 constructions. A similar calculation indicates that, with a total of 1400 chain conformers, the second design set shows more conformational complexity.

Intermediate Results

It is instructive to see the effect of dead-end elimination on the lists of amino acid residues.

TABLE

| Residue | Number of rotamers | | Amino Acids | |
|---|---|---|---|---|
| Position | Before | After | Before | After |

Dead-end elimination, modeling set CORE

| 9 | 9 | 5 | (AVILFW) | AVIL |
|---|---|---|---|---|
| 11 | 6 | 3 | ″ | AVI |
| 23 | 8 | 4 | ″ | VIL |
| 25 | 9 | 6 | ″ | AVIL |
| 31 | 15 | 5 | ″ | LI |
| 33 | 13 | 3 | ″ | LI |
| 44 | 8 | 3 | ″ | AVI |
| 53 | 9 | 6 | ″ | AVILF |
| 58 | 15 | 6 | | AVLF |
| | Number of conformations | | Number of sequences | |
| | 10^{8.9} | 10^{5.8} | 10^{7.0} | 10^{4.5} |

Dead-end elimination, modeling set SURFACE

| 46 | 61 | 1 | (AVILG | V |
|---|---|---|---|---|
| 47 | 96 | 1 | DNSTEQ | G |
| 48 | 153 | 1 | KRYW) | S |
| 49 | 136 | 1 | | K |
| | Number of conformations | | Number of sequences | |
| | 10^{8.1} | 1 | 10^{4.7} | 1 |

Similarly, we may observe the effect of the Mean Field Approximation on the distribution of conformers.

TABLE

| | | | | |
|---|---|---|---|---|
| Tempera- | Number | | | |
| ture | of | Absolute entropy | Weighted Score | GMEC |
| (K) | cycles | (kcal mol⁻¹) | (kcal mol⁻¹) | (kcal mol⁻¹) |

Mean field approximation, modeling set CORE (sequence IVIILLVIV
with 2520 conformations)

| | | | | |
|---|---|---|---|---|
| 1073 | 15 | 9.04 | −86.76 | |
| 973 | 23 | 7.98 | −87.04 | |
| 573 | 58 | 3.88 | −88.49 | |
| 473 | 67 | 2.98 | −88.91 | −70.77 |
| 373 | 77 | 2.29 | −89.27 | |
| 303 | 86 | 2.01 | −89.42 | |

Mean field approximation, modeling set SURFACE (sequence VGSK
with 768 conformations)

| | | | | |
|---|---|---|---|---|
| 1073 | 16 | 12.60 | 5.11 | |
| 973 | 23 | 11.26 | 4.94 | |
| 873 | 30 | 9.93 | 4.75 | |
| 473 | 63 | 4.56 | 3.66 | 2.06 |
| 373 | 72 | 3.26 | 3.28 | |
| 303 | 81 | 2.38 | 2.99 | |

Output Sequences

Sample sequences along with their solution scores as output from the program are as follows:

| CORE | | | SURFACE | | |
|---|---|---|---|---|---|
| VAVMLLVVV | −76.6 | (Wild Type) | VNDR | −6.6 | (Wild Type) |
| LVIVLLVIV | −81.8 | | VGSK | −28.0 | |
| VVIILLVIV | −81.8 | | | | |
| IVIILLVVV | −81.9 | | | | |
| LIIVLLVIV | −82.0 | | | | |
| IVVILLVIV | −82.8 | | | | |
| IIIVLLVIV | −82.8 | | | | |
| IVLILLVIV | −82.9 | | | | |
| LVIILLVIV | −83.2 | | | | |
| IVIILLVIV | −84.4 | | | | |

We note that the solution scores of the best solutions are all somewhat lower than the score of the “wild type” sequence. The accompanying figure shows the distribution of solution scores for approximately 1600 considered solutions.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method for choosing a set of building blocks in a target macromolecule, the method comprising:
(a) specifying at least one substitute for each building block in said set of building blocks;
(b) determining, for each said substitute, at least one candidate conformer;
(c) substituting coordinates of each said candidate conformer or portion thereof for coordinates of a corresponding building block or portion thereof in an atomic structure of said target macromolecule; and
(d) minimizing the value of a calculated solution score by adjusting the geometry of each said candidate conformer or portion thereof in order to obtain a solution structure; and
(e) determining whether said solution structure has a calculated solution score that is lower than a threshold value.

2. The method of claim 1 wherein (i) said macromolecule is a peptide or protein; (ii) said building blocks are amino acid residues; and (iii) each candidate conformer is a side chain rotamer selected from plurality of side chain rotamers.

3. The method of claim 2 where said calculated solution score comprises a difference between a first value corresponding to said solution structure and a second value corresponding to a reference structure.

4. The method of claim 3, wherein said first value corresponding to said solution structure accounts for (i) interactions between said side chain rotamer and said atomic structure and (ii) a sum of interactions between all pairs of all possible side chain rotamers.

5. The method of claim 3, wherein said reference structure is a denatured state of said solution structure.

6. The method of claim 4, further comprising a step of rejecting a side chain rotamer when the value of said interactions between said side chain rotamer and said atomic structure is greater than a threshold value.

7. The method of claim 2, wherein the dihedral angles of said side chain rotamers are optimized in step (d).

8. The method of claim 2, wherein the positions of all main chain atoms of said atomic structure, and the positions of all atoms in amino acid side chains that are not included in said set of building blocks are held fixed in said atomic structure.

9. The method of claim 7, wherein the positions of all atoms in amino acid side chains that are not included in said set of building blocks are allowed to vary whilst the dihedral angles of said rotamer are optimized.

10. The method of claim 7, wherein the positions of all main chain atoms of said atomic structure are allowed to vary whilst the dihedral angles of said rotamer are optimized.

11. The method of claim 2, wherein said plurality of side chain rotamers is a library of predetermined rotamer conformations.

12. The method of claim 2, wherein said plurality of side chain rotamers is derived from a continuous distribution of conformations.

13. The method of claim 1, wherein:
(i) said atomic structure includes a representation of all building blocks in said set of building blocks; and
(ii) said atomic structure was determined by a method selected from the group consisting of x-ray crystallography, nuclear magnetic resonance spectroscopy, electron microscopy, homology modeling, and ab initio modeling.

14. The method of claim 1, wherein said atomic structure is an X-ray crystal structure of a portion of said macromolecule that comprises said set of building blocks.

15. The method of claim 14, wherein said X-ray crystal structure was determined at a resolution of less than 4.0 Angstroms.

16. The method of claim 2, wherein said calculated solution score is calculated using an empirical scoring function.

17. The method of claim 16, wherein said empirical scoring function is a sum of energy terms, comprising an energy of said atomic structure held in a fixed geometry, an intrinsic energy of interaction between a candidate side chain rotamer and said atomic structure held in a fixed geometry and a pairwise energy of interaction between possible pairs of side chain rotamers in said set of building blocks.

18. The method of claim 17 wherein said intrinsic energy of interaction is computed as:
$$Intrinsic Energy = \{ \begin{matrix}either {E}_{\begin{matrix}fixed structure - \\ best side chain ( i )\end{matrix}} \\ or {\sum }_{\begin{matrix}rotamers \\ r\end{matrix}} {w}_{i , r} {E}_{\begin{matrix}fixed structure - \\ side chain ( i , r )\end{matrix}}\end{matrix}$$
where E_{fixed structure-side chain(i)} is an energy of interaction between the atomic structure held in a fixed geometry and side chain i.

19. The method of claim 17, wherein said pairwise energy of interaction is computed as:
$$Pairwise Energy = \{ \begin{matrix}either {E}_{\begin{matrix}best side chain ( i ) - \\ best side chain ( j )\end{matrix}} \\ or {\sum }_{\begin{matrix}rotamers r \\ or residue i\end{matrix}} {\sum }_{\begin{matrix}rotamers s \\ or residue j\end{matrix}} {w}_{i , r} {w}_{j , s} {E}_{\begin{matrix}side chain ( i , r ) - \\ side chain ( j , s )\end{matrix}}\end{matrix}$$
where E_{side chain(i)-side chain(j)} is the energy of interaction between side chain rotamer i and side chain rotamer j and ω is a weight.

20. The method of claim 17, wherein the energy of said atomic structure held in a fixed geometry comprises at least one energy term selected from the group consisting of a molecular mechanics potential, a salvation energy, an empirical penalty function, and an entropic contribution.

21. The method of claim 20, wherein the energy of said atomic structure held in a fixed geometry comprises a sum of terms whose coefficients are individually adjustable weighting factors.

22. The method of claim 20, wherein said molecular mechanics potential comprises at least one energy term selected from the group consisting of bond length vibrations, bond angle bends, the hydrogen bond energy between pairs of hydrogen bond donor and acceptor atoms, an electrostatic interaction energy between pairs of charged atoms, and a van der Waals interaction energy between pairs of non-bonded atoms in said atomic structure.

23. The method of claim 22, wherein the van der Waals term is expressed as a sum:
Σ_{nonbonded i,j} (Aᵢⱼ/rᵢⱼ¹²−Bᵢⱼ/rᵢⱼ⁶)
wherein said sum runs over all possible non-bonded atom pairs i and j from said atomic structure held in a fixed geometry.

24. The method of claim 22, wherein the hydrogen bonding energy between pairs of hydrogen bond donor and acceptor atoms is expressed as a sum:
Σ_{H-bonds ij} (A′ᵢⱼ/rᵢⱼ¹²−B′ᵢⱼ/tᵢⱼ¹⁰)
wherein said sum runs over all hydrogen bonds in said atomic structure held in a fixed geometry and atoms i and j are donor and acceptor atoms participating in each of said hydrogen bonds.

25. The method of claim 22, wherein said electrostatic interaction energy between pairs of charged atoms is expressed as a sum:
Σ_{charges i,j}qᵢqⱼe²/4πε₀εᵣrᵢⱼ
wherein the said sum runs over all pairs of charged atoms i, and j in said atomic structure held in a fixed geometry whose respective charges are qᵢ and qⱼ.

26. The method of claim 20, wherein said entropic contribution comprises at least one term selected from the group consisting of a main chain entropy term, a side chain rotation entropy term and a side chain vibration entropy term.

27. The method of claim 26 wherein said main chain entropy term is:
$$- {w}_{main chain}^{entropy} {RT}_{phys} {\sum }_{all residues i} ln \frac{{\sum }_{\begin{matrix}subspaces {φψ}_{20 ° \times 20 °} \\ close to {φ}_{i} {ψ}_{i}\end{matrix}} {w}_{{φψ}_{20 ° \times 20 °} {N}_{{φψ}_{20 ° \times 20 °}}^{amino acid i}}}{{N}_{all φψ}^{amino acid i}}$$
wherein ω is a coefficient, T is temperature, and N is number of side chain rotamers found within a specific range of φ, ψ angles.

28. The method of claim 26 wherein said side chain rotation entropy term is calculated as
$$- {w}_{side chain}^{entropy} {T}_{phys} {\sum }_{all residues i} ( - R {\sum }_{\begin{matrix}all rotamers r \\ of residue i\end{matrix}} {w}_{r} ln {w}_{r} )$$
wherein ω is a coefficient, T is a temperature, and ωᵣ is obtained from a partition function.

29. The method of claim 26, wherein said side chain vibration entropy term is:
$$- {w}_{sidechain}^{vibration} {T}_{phys} {\sum }_{allresidues i} {\sum }_{\begin{matrix}all rotamers r \\ of residue i\end{matrix}} {w}_{r} ( - R {\sum }_{\begin{matrix}allsub \text{-} rotamerss \\ ofrotamer r\end{matrix}} {w}_{s} ln {w}_{s} )$$
wherein ω is a coefficient and ωᵣ is obtained from a partition function.

30. The method of claim 20, wherein said salvation energy is calculated as
$${w}^{solvaation} {\sum }_{atoms i} {σ}_{i} {ASA}_{i}$$
wherein σ is an atom-specific parameter, ω is a coefficient and ASA₁ is an accessible surface area of atom i and atom i is in said atomic structure.

31. The method of claim 30, wherein said atom-specific parameters reflect the properties of a solvent selected from the group consisting of water and an organic solvent.

32. The method of claim 31 wherein the organic solvent is selected from the group consisting of methanol, methylamine, and dimethyl sulphoxide.

33. The method of claim 20, wherein said empirical penalty function is calculated as
$$- {w}^{stat} {RT}_{stat} {\sum }_{all residues i} ln {P}_{amino acid i}^{stat}$$
wherein P is a term representing a probability of occurrence of a given amino acid in nature.

34. The method of claim 5, wherein said reference structure comprises said side chain rotamer substituted for a side chain in an alanine based penta-peptide.

35. The method of claim 5 wherein said reference structure comprises said side chain rotamer embedded in a fragment of protein taken from an atomic structure of a naturally occurring protein or an ensemble of fragments of protein, the populations of which are determined either from populations in the naturally occurring proteins or from computations establishing the potential energy of each fragment and integrating them into a partition function.

36. The method of claim 26, wherein said reference structure is a denatured state of said atomic structure and said side chain vibration entropy term in said reference structure is modelled as a uniform distribution of sub-rotamer conformations.

37. The method of claim 18, wherein said intrinsic energy of interaction comprises at least one energy term selected from the group consisting of a molecular mechanics energy term, asolvation energy term, and in entropic contribution.

38. The method of claim 37, wherein said intrinsic energy of interaction comprises a sum of terms whose coefficients are individually adjustable weighting factors.

39. The method of claim 37, wherein said molecular mechanics energy term comprises at least one term selected from the group consisting of the van der Waals energy between pairs of non-bonded atoms, the hydrogen bond energy between pairs of hydrogen bond donor and acceptor atoms, and the electrostatic interaction energy between pairs of charged atoms.

40. The method of claim 39, wherein the van der Waals energy between pairs of non-bonded atoms is:
Σ_{nonbonded i,j} (Aᵢⱼ/rᵢⱼ¹²−Bᵢⱼ/rᵢⱼ⁶)
where i and j represent all possible atom pairs comprising atoms i of said atomic structure held in a fixed geometry and j of said side chain rotamer.

41. The method of claim 39, wherein the hydrogen bonding energy is:
Σ_{H-bonds ij}(A′ᵢⱼ/rᵢⱼ¹²−B′ᵢⱼ/rᵢⱼ¹⁰)
wherein said sum runs over all hydrogen bonds between said atomic structure held in a fixed geometry and said side chain rotamer, and atoms i and j are donor and acceptor atoms participating in each of said hydrogen bonds.

42. The method of claim 39, wherein the electrostatic energy between pairs of charged atoms is:
Σ_{charges ij}qᵢqⱼe²/4πε₀εᵣrᵢⱼ
wherein said sum runs over all pairs of charged atoms such that atom i is found in said atomic structure and j is found in said side chain rotamer and whose respective charges are qᵢ and qⱼ.

43. The method of claim 37, wherein said entropic contribution comprises at least one term selected from the group consisting of a main chain entropy term and a side chain vibration entropy term.

44. The method of claim 43, wherein said main chain entropy term is:
$$- {w}_{mainchain}^{entropy} {RT}_{phys} ln \frac{{\sum }_{\begin{matrix}subspaces {φψ}_{20 ° \times 20 °} \\ close to {φ}_{i} {ψ}_{i}\end{matrix}} {w}_{{φψ}_{20 ° \times 20 °}} {N}_{{φψ}_{20 ° \times 20 °}}^{\begin{matrix}residue \\ type\end{matrix}}}{{N}_{all φψ}^{\begin{matrix}residue \\ type\end{matrix}}}$$
wherein ω is a coefficient, ω_{φψ} is obtained from the partition function and N is a number of rotamers found in a given range of dihedral angles.

45. The method of claim 43, wherein said side chain vibration entropy term is:
$$- {w}_{side chain}^{vibration} {T}_{phys} [ {( - R {\sum }_{sub \text{-} rotamers s} {w}_{s} ln {w}_{s} )}_{\begin{matrix}target \\ structure\end{matrix}} - {VIB}_{\begin{matrix}reference \\ frame\end{matrix}}^{\begin{matrix}residue \\ type\end{matrix}} ] .$$

46. The method of claim 37, wherein said solvation term is:
$${w}^{solvation} {\sum }_{atoms i of side chain} {σ}_{i} [ {( {ASA}_{i} )}_{\begin{matrix}reference \\ frame \end{matrix}} - {( {ASA}_{i} )}_{\begin{matrix}target \\ structure\end{matrix}} ] .$$

47. The method of claim 19, wherein said pairwise energy of interaction comprises at least one energy term selected from the group consisting of a molecular mechanics term, a salvation energy term, a penalty term, and an entropic contribution.

48. The method of claim 47, wherein said pairwise energy of interaction comprises a sum of terms whose coefficients are individually adjustable weighting factors.

49. The method of claim 47, wherein said molecular mechanics term comprises at least one term selected from the group consisting of a van der Waals energy term, a hydrogen bond energy term and an electrostatic interaction energy term.

50. The method of claim 49, wherein the van der Waals energy term is:
Σ_{nonbonded ij}(Aᵢⱼ/rᵢⱼ¹²−Bᵢⱼ/rᵢⱼ⁶)
and i and j represent all possible atom pairs comprising atoms i from the first side chain rotamer of one of said pairs of side chain rotamers and atoms j from the second side chain rotamer of one of said pairs of side chain rotamers.

51. The method of claim 49, wherein the hydrogen bonding energy term is:
Σ_{H-bonds ij}(A′ᵢⱼ/rᵢⱼ¹²−B′ᵢⱼ/rᵢⱼ¹⁰).
and i and j represent all possible hydrogen bonds between atom pairs comprising atoms i from the first side chain rotamer of one of said pairs of side chain rotamers and atoms j from the second side chain rotamer of one of said pairs of side chain rotamers.

52. The method of claim 49, wherein the electrostatic interaction energy term is:
Σ_{charges ij}qᵢqⱼe²/4πε₀εᵣrᵢⱼ.
and i and j represent all possible pairs of charged atoms comprising atoms i, with charge q₁, from the first side chain rotamer of one of said pairs of side chain rotamers and atoms j, with charge qⱼ, from the second side chain rotamer of one of said pairs of side chain rotamers.

53. The method of claim 47 wherein said entropic contribution comprises a side chain vibration entropy term.

54. The method of claim 53, wherein said side chain vibration entropy term is calculated according to an energy-based distribution of sub-rotamers.

55. The method of claim 53 wherein said side chain vibrational entropy term is:
$$- {w}_{Pairwise}^{vibration} {T}_{phys} λ [ {( - R {\sum }_{\begin{matrix}sub \text{-} rotamers {A}_{s} and {B}_{s} \\ of rotamer pair AB\end{matrix}} {w}_{{A}_{s} {B}_{s}} ln {w}_{{A}_{s} {B}_{s}} )}_{\begin{matrix}rotamer pair AB \\ in target structure\end{matrix}} - {( - R {\sum }_{\begin{matrix}sub \text{-} rotamers {A}_{s} \\ of rotamer A\end{matrix}} {w}_{{A}_{s}} ln {w}_{{A}_{s}} )}_{\begin{matrix}only rotamer A \\ in target structure\end{matrix}} - {( - R {\sum }_{\begin{matrix}sub \text{-} rotamers {B}_{x} \\ of rotamer B\end{matrix}} {w}_{{B}_{s}} ln {w}_{{B}_{s}} )}_{\begin{matrix}only rotamer B \\ in target structure\end{matrix}} ]$$

56. The method of claim 55, wherein the weights, ωₛ, of the sub-rotamers in said side chain vibration entropy term are obtained by means of a partition function.

57. The method of claim 47, wherein said solvation energy term is calculated as a difference between an accessible surface area of the side chain in the reference state and the measured accessible surface area of the side chain conformer substituted into the atomic structure in accordance with step (c) of claim 2 and the reference structure is a denatured form of the macromolecule:

58. The method of claim 47, wherein said solvation term is calculated as a difference between a measured accessible surface area of each side chain conformer substituted, separately, into the atomic structure, in accordance with step (c) of claim 2, and the measured accessible surface area of each side chain substituted together into the target atomic structure in accordance with step (c) of claim 2.
$$+ {w}^{solvation} {\sum }_{\begin{matrix}atoms i of residue A or B \\ of residue pair AB\end{matrix}} {σ}_{i} λ [ \begin{matrix}{( {ASA}_{i} )}_{\begin{matrix}only residue A or B \\ in target structure\end{matrix}} \\ - {( {ASA}_{i} )}_{\begin{matrix}only residue AB \\ in target structure\end{matrix}}\end{matrix} ]$$

59. The method of claim 47, wherein said penalty term is:
$$- {w}_{Pairwise}^{stat} {RT}_{stat} ln {P}_{\begin{matrix}residue \\ pair\end{matrix}}^{state}$$
wherein P is a probability of occurrence of the amino acid pair in question.

60. The method of claim 7, wherein the dihedral angles of said side chain rotamer are optimized by minimizing the molecular mechanics terms of the intrinsic energy function of claim 37, and the molecular mechanics terms of the pairwise energy function of claim 47.

61. The method of claim 60, wherein the dihedral angles of said side chain rotamer are optimized in the space of internal rotations of said rotamers by an algorithm selected from the group consisting of least squares, steepest descents, quasi-Newtonian, and simulated annealing.

62. The method of claim 60, wherein the dihedral angles of said side chain rotamer are optimized by averaging over the values measured by sampling sub-rotamer configurations of the side chain rotamers, generating said sub-rotamer configurations by sampling a range of dihedral angles by stepping each dihedral angle in said side chain rotamer by a predetermined step size for a number of steps and, selecting said sub-rotamers whose contribution to the calculated solution score is highest.

63. The method of claim 62, wherein the predetermined step size and the number of steps sampled is determined by an amino acid type of the side chain rotamer.

64. The method of claim 6, wherein the side chain rotamers which are not rejected and that have a probability smaller than a predetermined probability threshold are rejected.

65. The method of claim 64, wherein said probability is calculated from a partition function over all possible rotamers of a particular residue type, using their energy of interaction with a fixed portion of the atomic structure as calculated by the intrinsic energy of interaction of claim 37.

66. The method of claim 2, wherein each candidate conformer specified in step (b) is a D-enantiomer selected from the group consisting of: glycine, alanine, valine, leucine, isoleucine, glutamic acid, aspartic acid, asparagine, glutamine, proline, phenylalanine, tyrosile, serine, threonine, lysine, arginine, histidine, cysteine, tryptophan, and methionine.

67. The method of claim 2, wherein each candidate conformer specified in step (b) is an L-enantiomer selected from the group consisting of: glycine, alanine, valine, leucine, isoleucine, glutamic acid, aspartic acid, asparagine, glutamine, proline, phenylalanine, tyrosine, serine, threonine, lysine, arginine, histidine, cysteine, tryptophan, and methionine.

68. The method of claim 2, wherein each candidate conformer specified in step (b) is selected from the group consisting of the L- and D-enantiomers of amino acids including, but not limited to, norvaline, beta-alanine, and tartrine.

69. The method of claim 11, wherein said library of predetermined rotamer conformations is constructed by a method comprising:
(a) tabulating, for each of the twenty naturally occurring amino acids, a statistical distribution of observed amino acid side chain dihedral angles in a set of crystallographically determined protein structures;
(b) determining a Gaussian distribution of observed amino acid side chain dihedral angles tabulated in (a); and
(c) constructing amino acid side chain rotamers for each of the twenty naturally occurring amino acids using all combinations of Gaussian peaks around each amino acid side chain dihedral angle derived in (b).

70. The method of claim 11, wherein said library of predetermined rotamer conformations is constructed ab initio, by a method comprising: computing portions of vibration-rotation potential energy surfaces of said side chain rotamers and determining, through exhaustive sampling, dihedral angles at which minima are found on said vibration-rotation potential energy surfaces.

71. The method of claim 2, further comprising the step of storing all side chain rotamers having a calculated solution score that is lower than said first threshold value and in an array and eliminating a subset of side chain rotamers from said array using dead-end elimination where energy terms are partitioned according to:
$$Template Energy + {\sum }_{{residues}_{i}} Intrinsic Energy + {\sum }_{{residues}_{i}} {\sum }_{{residues}_{j > i}} Pairwise Energy .$$

72. The method of claim 71, wherein dead-end elimination comprises elimination of a side chain rotamer r, of residue i, when the inequality
$${E}_{i}^{{r}^{template}} - {E}_{{i}_{t}}^{template} + {\sum }_{j \neq 1} {min}_{s} ( {E}_{{i}_{r} , {j}_{s}}^{pairwise} - {E}_{{i}_{t} , {j}_{s}}^{pairwise} ) > 0$$
is true.

73. The method of claim 2, further comprising the step of eliminating a side chain rotamer pair using dead-end elimination,,wherein a side chain rotamer pair comprises a first side chain rotamer corresponding to a first building block in said set of building blocks and a second side chain rotamer corresponding to a second building block in said set of building blocks.

74. The method of claim 73, wherein dead-end elimination comprises eliminating a side chain rotamer pair that consists of rotamer r, of residue i, and rotamer s, of residue j, when the inequalities:
and
$${E}_{( {i}_{r} , {j}_{s} )} + {\sum }_{k \neq i , j} {min}_{t} ( {E}_{( {i}_{r} , {j}_{s} ) , {k}_{t}} ) > {E}_{( {i}_{u} , {j}_{v} )} + {\sum }_{k \neq i , j} {max}_{t} ( {E}_{( {i}_{u} , {j}_{v} ) , {k}_{t}} ) and$$
$${E}_{( {i}_{r} , {j}_{s} )} - {E}_{( {l}_{u} , {j}_{v} )} + {\sum }_{k \neq i , j} {min}_{i} ( {E}_{( {i}_{r} , {j}_{s} ) , {k}_{i}} - {E}_{( {i}_{u} , {j}_{v} ) , {k}_{t}} ) > 0$$
are true.

75. The method of claim 71, wherein the eliminating step is repeated until no additional side chain rotamers are eliminated from said array by the process of dead-end elimination.

76. The method of claim 73, wherein the eliminating step is repeated until no additional side chain rotamer pairs are eliminated from said array by the process of dead-end elimination.

77. The method of claim 2, wherein the calculated solution score additionally comprises an entropy term that is determined by a difference between (i) an entropy of a side chain rotamer in the solution when substituted into the atomic structure and (ii) an entropy of the side chain rotamer in the solution when in a denatured state.

78. The method of claim 77 wherein entropy contributions of said side chain rotamers are derived from experimental or empirical data.

79. The method of claim 77, wherein the entropy term is computed using an iterative method.

80. The method of claim 79, wherein the entropy term is computed using mean field theory,

81. The method of claim 80, wherein the contribution of the rotamers to the entropy may be split into intrinsic and pairwise terms.

82. The method of claim 2 wherein said calculated solution score comprises a difference between a first value corresponding to said solution structure and a second value corresponding to a weighted average over a plurality of reference structures.

83. The method of claim 1 wherein said target macromolecule consists of a plurality of structures at least one of which is represented by an atomic structure.

84. A computer program product for use in conjunction with a computer, the computer program product comprising a computer readable storage medium and a computer program mechanism embedded therein, the computer program mechanism comprising an optimizer module configured to work with a set of building blocks in a macromolecule, the computer program mechanism, upon receiving as input a set of building blocks:
(a) determining at least one substitute for each building block in said set of building blocks:
(b) determining, for each said substitute, at least one candidate conformer;
(c) substituting coordinates of each said candidate conformer or portion thereof for coordinates of a building block or portion thereof in an atomic structure of said target macromolecule; and
(d) minimizing the value of a calculated solution score by adjusting the geometry of each said candidate conformer or portion thereof in order to obtain a solution structure; and
(e) determining whether said solution structure has a solution score that is lower than a first threshold value.

85. The computer program product of claim 84, wherein:
(i) said macromolecule is a peptide or protein;
(ii) said building blocks are amino acid residues; and
(iii) each candidate conformer is a side chain rotamer selected from a plurality of side chain rotamers.

86. The computer program product of claim 85 wherein said calculated solution score comprises a difference between a first value corresponding to said solution structure and a second value corresponding to a reference structure

87. The computer program product of claim 86, wherein said first value corresponding to said solution structure accounts for (i, interactions between said side chain rotamer and said atomic structure and (ii) a sum of interactions between all pairs of all possible side chain rotamers.

88. The computer program product of claim 87, further comprising a step of rejecting a side chain rotamer when the value of said interactions between said side chain rotamer and said atomic structure is greater than a threshold value.

89. The computer program product of claim 86, wherein said reference structure is a denatured state of said solution structure.

90. The computer program product of claim 85, wherein the dihedral angles of said side chain rotamer are optimized in step (d).

91. The computer program product of claim 85, wherein the positions of all main chain atoms of said atomic structure, and the positions of all atoms in amino acid side chains that are not included in said set of building blocks are held fixed in said atomic structure.

92. The computer program product of claim 90, wherein the positions of all atoms in amino acid side chains that are not included in said set of building blocks are allowed to vary whilst the dihedral angles of said side chain rotamer are optimized.

93. The computer program product of claim 90, wherein the positions of all main chain atoms of said atomic structure are allowed to vary whilst the dihedral angles of said side chain rotamer are optimized.

94. The computer program product of claim 85, wherein said plurality of conformers is a library of predetermined side chain rotamer conformations.

95. The computer program product of claim 85, wherein at least one side chain conformer in said plurality of side chain rotamers is derived from a continuous distribution of conformations.

96. The computer program product of claim 84, wherein:
(i) said atomic structure includes a representation of all building blocks in said set of building blocks; and
(ii) said atomic structure was determined by a method selected from the group consisting of x-ray crystallography, nuclear magnetic resonance spectroscopy, electron microscopy, homology modeling, and ab initio modeling.

97. The computer program product of claim 84, wherein said atomic structure is an X-ray crystal structure of a portion of said macromolecule that comprises said set of building blocks.

98. The computer program product of claim 97, wherein said X-ray crystal structure was determined at a resolution of less than 4.0 Angstroms.

99. The computer program product of claim 85, wherein said solution score is calculated using an empirical scoring function.

100. The computer program product of claim 99, wherein said empirical scoring function is a sum of energy terms, comprising an energy of said atomic structure held in a fixed geometry, an intrinsic energy of interaction between a side chain rotamer and said atomic structure held in a fixed geometry and a pairwise energy of interaction between possible pairs of side chain rotamers in said set of building blocks.

101. The computer program product of claim 100, wherein the energy of said atomic structure comprises at least one energy term selected from the group consisting of a molecular mechanics potential, a solvation energy, an empirical penalty function, and an entropic contribution.

102. The computer program product of claim 101, wherein the energy of said atomic structure comprises a sum of terms whose coefficients are individually adjustable weighting factors.

103. The computer program product of claim 102, wherein said molecular mechanics potential comprises at least one energy term selected from the group consisting of bond length vibrations, bond angle bends, the hydrogen bond energy between pairs of hydrogen bond donor and acceptor atoms, an electrostatic interaction energy between pairs of charged atoms, and a van der Waals interaction energy between pairs of non-bonded atoms in said atomic structure.

104. The computer program product of claim 101, wherein said entropic contribution comprises at least one term selected from the group consisting of a main chain entropy term, a side chain rotation entropy term and a side chain vibration entropy term.

105. The computer program product of claim 89, wherein said reference structure comprises said side chain rotamer embedded in an alanine based penta-peptide.

106. The computer program product of claim 89, wherein said reference structure comprises said side chain rotamer embedded in a fragment of protein taken from an atomic structure of a naturally occurring protein or an ensemble of fragments of protein, the populations of which are determined either from populations in the naturally occurring proteins or from computations establishing the potential energy of each fragment and integrating them into a partition function.

107. The computer program product of claim 88, wherein the side chain rotamers which are not rejected and that have a probability smaller than a predetermined probability threshold are rejected.

108. The computer program product of claim 94, wherein said library of predetermined rotamer conformations is constructed by a method comprising:
(a) Tabulating, for each of the twenty naturally occurring amino acids, a statistical distribution of observed amino acid side chain dihedral angles in a set of crystallographically determined protein structures;
(b) determining a Gaussian distribution of observed amino acid side chain dihedral angles tabulated in (a); and
(c) constructing amino acid side chain rotamers for each of the twenty naturally occurring amino acids using all combinations of Gaussian peaks around each amino acid side chain dihedral angle derived in (b).

109. The computer program product of claim 94 wherein said library of predetermined rotamer conformations is constructed ab initio, by a method comprising: computing portions of vibration-rotation potential energy surfaces of said side chain rotamers and determining, through exhaustive sampling, dihedral angles at which minima are found on said vibration-rotation potential energy surfaces.

110. The computer program product of claim 85, further comprising the step of storing all side chain rotamers having a solution score that is lower than said first threshold value and in an array and eliminating a subset of side chain rotamers from said array using dead-end elimination where energy terms are partitioned according to:
$$Template Energy + {\sum }_{{residues}_{i}} Intrinsic Energy + {\sum }_{{residues}_{i}} {\sum }_{{residues}_{j > i}} Pairwise Energy .$$

111. The computer program product of claim 110, wherein dead-end elimination comprises elimination of a side chain rotamer r, of residue i, when the inequality
$${E}_{i}^{{r}^{template}} - {E}_{{i}_{t}}^{template} + {\sum }_{j \neq 1} {min}_{s} ( {E}_{{i}_{r} , {j}_{s}}^{pairwise} - {E}_{{i}_{t} , {j}_{s}}^{pairwise} ) > 0$$
is true.

112. The computer program product of claim 85, further comprising the step of eliminating a side chain rotamer pair using dead-end elimination, wherein a side chain rotamer pair comprises a first side chain rotamer representing a first building block in said set of building blocks and a second side chain rotamer representing a second building block in said set of building blocks.

113. The computer program product of claim 112, wherein dead-end elimination comprises eliminating a side chain rotamer pair that consists of rotamer r, of residue i, and rotamer s, of residue j, when the inequalities:
$${E}_{( {i}_{r} , {j}_{s} )} + {\sum }_{k \neq i , j} {min}_{t} ( {E}_{( {i}_{r} , {j}_{s} ) , {k}_{t}} ) > {E}_{( {i}_{u} , {j}_{v} )} + {\sum }_{k \neq i , j} {max}_{t} ( {E}_{( {i}_{u} , {j}_{v} ) , {k}_{t}} ) and$$
$${E}_{( {i}_{r} , {j}_{s} )} - {E}_{( {l}_{u} , {j}_{v} )} + {\sum }_{k \neq i , j} {min}_{i} ( {E}_{( {i}_{r} , {j}_{s} ) , {k}_{i}} - {E}_{( {i}_{u} , {j}_{v} ) , {k}_{t}} ) > 0$$
and
are true.

114. The computer program product of claim 110, wherein the eliminating step is repeated until no additional side chain rotamers are eliminated from said array by the process of dead-end elimination.

115. The computer program product of claim 112, wherein the eliminating step is repeated until no additional side chain rotamer pairs are eliminated from said array by the process of dead-end elimination.

116. The computer program product of claim 85, wherein the solution score additionally comprises an entropy term that is determined by a difference between (i) an entropy of a side chain rotamer in the solution when substituted into the atomic structure and (ii) an entropy of the side chain rotamer in the solution when in a denatured state.

117. The computer program product of claim 116, wherein entropy contributions of said side chain rotamers are derived from experimental or empirical data.

118. The computer program product of claim 116, wherein the entropy term is computed using an iterative method.

119. The computer program product of claim 118, wherein the entropy term is computed using mean field theory.

120. The computer program product of claim 119, wherein the contribution of the rotamers to the entropy may be split into intrinsic and pairwise terms.

121. A system for choosing a set of building blocks in a macromolecule comprising:
a central processing unit;
an input device for inputting requests;
an output device;
a memory;
at least one bus connected to the central processing unit, the memory, the input device, and the output device;
the memory storing an computer program mechanism comprising an optimizer module configured to optimize the set of building blocks, the computer program mechanism, upon receiving a request to optimize the set of building blocks,
(a) determining at least one substitute for each building block in said set of building blocks:
(b) determining, for each substitute, at least one candidate conformer;
(c) substituting coordinates of said candidate conformer or portion thereof for coordinates of a corresponding building block or portion thereof in an atomic structure of said target macromolecule; and
(d) minimizing the value of a calculated solution score by adjusting the geometry of the candidate conformer in order to obtain a solution structure; and
(e) determining whether said solution structure has a solution score that is lower than a threshold value.

122. The system of claim 121, wherein:
(i) said macromolecule is a peptide or protein;
(ii) said building blocks are amino acid residues; and
(iii) each candidate conformer is a side chain rotamer selected from a plurality of side chain rotamers.

123. The system of claim 122 where said calculated solution score comprises a difference between a first value corresponding to said solution structure and a second value corresponding to a reference structure.

124. The system of claim 123, wherein said first value corresponding to said solution structure accounts for (i) interaction between said side chain rotamer and said atomic structure and (ii) a sum of interactions between all pairs of all possible side chain rotamers.

125. The system of claim 124, further comprising a step of rejecting a side chain rotamer when the value of said interactions between said side chain rotamer and said atomic structure is greater than a threshold value.

126. The system of claim 123, wherein said reference structure is a denatured state of said solution structure.

127. The system of claim 122, additionally comprising a step wherein the dihedral angles of said side chain rotamer are optimized in step (d).

128. The system of claim 122, wherein the positions of all main chain atoms of said atomic structure, and the positions of all atoms in amino acid side chains that are not included in said set of building blocks are held fixed in said atomic structure.

129. The system of claim 127, wherein the positions of all atoms in amino acid side chains that are not included in said set of building blocks are allowed to vary whilst the dihedral angles of said rotamer are optimized.

130. The system of claim 127, wherein the positions of all main chain atoms of said atomic structure are allowed to vary whilst the dihedral angles of said rotamer are optimized.

131. The system of claim 122, wherein said plurality of conformers is a library of predetermined rotamer conformations.

132. The system of claim 122, wherein at least one side chain rotamer in said plurality of side chain rotamers is derived from a continuous distribution of conformations.

133. The system of claim 121, wherein:
(i) said atomic structure includes a representation of all building blocks in said set of building blocks; and
(ii) said atomic structure was determined by a method selected from the group consisting of x-ray crystallography, nuclear magnetic resonance spectroscopy, electron microscopy, homology modeling, and ab initio modeling.

134. The system of claim 121, wherein said atomic structure is an X-ray crystal structure of a portion of said macromolecule that comprises said set of building blocks.

135. The system of claim 124, wherein said X-ray crystal structure was determined at a resolution of less than 4.0 Angstroms.

136. The system of claim 122, wherein said calculated solution score is obtained using an empirical scoring function.

137. The system of claim 136, wherein said empirical scoring function is a sum of energy terms, comprising an energy of said atomic structure held in a fixed geometry, an intrinsic energy of interaction between a side chain rotamer and said atomic structure held in a fixed geometry and a pairwise energy of interaction between possible pairs of side chain rotamers in said set of building blocks.

138. The system of claim 137, wherein the energy of said atomic structure comprises at least one energy term selected from the group consisting of a molecular mechanics potential, a solvation energy, an empirical penalty function, and an entropic contribution.

139. The system of claim 138, wherein the energy of said atomic structure comprises a sum of terms whose coefficients are individually adjustable weighting factors.

140. The system of claim 139, wherein said molecular mechanics potential comprises at least one energy term selected from the group consisting of bond length vibrations, bond angle bends, the hydrogen bond energy between pairs of hydrogen bond donor and acceptor atoms, an electrostatic interaction energy between pairs of charged atoms, and a van der Waals interaction energy between pairs of non-bonded atoms in said atomic structure.

141. The system of claim 138, wherein said entropic contribution comprises at least one term selected from the group consisting of a main chain entropy term, a side chain rotation entropy term and a side chain vibration entropy term.

142. The system of claim 125, wherein said reference structure comprises said side chain rotamer substituted for a side chain rotamer in an alanine based penta-peptide.

143. The system of claim 125, wherein said reference structure comprises said side chain rotamer embedded in a fragment of protein taken from an atomic structure of a naturally occurring protein or an ensemble of fragments of protein, the populations of which are determined either from populations in the naturally occurring proteins or from computations establishing the potential energy of each fragment and integrating them into a partition function.

144. The system of claim 124, wherein the side chain rotamers which are not rejected in and that have a probability smaller than a predetermined probability threshold are rejected.

145. The system of claim 131, wherein said library of predetermined rotamer conformations is constructed by a method comprising:
(a) tabulating, for each of the twenty naturally occurring amino acids, a statistical distribution of observed amino acid side chain dihedral angles in a set of crystallographically determined protein structures;
(b) determining a Gaussian distribution of observed amino acid side chain dihedral angles tabulated in (a); and
(c) constructing amino acid side chain rotamers for each of the twenty naturally occurring amino acids using all combinations of Gaussian peaks around each amino acid side chain dihedral angle derived in (b).

146. The system of claim 131, wherein said library of predetermined rotamer conformations is constructed ab initio, by a method comprising: computing portions of vibration-rotation potential energy surfaces of said side chain rotamers and determining, through exhaustive sampling, dihedral angles at which minima are found on said vibration-rotation potential energy surfaces.

147. The system of claim 122, further comprising the step of storing all side chain rotamers having a solution score that is lower than said first threshold value and in an array and eliminating a subset of side chain rotamers from said array using dead-end elimination where energy terms are partitioned according to:
$$Template Energy + {\sum }_{{residues}_{i}} Intrinsic Energy + {\sum }_{{residues}_{i}} {\sum }_{{residues}_{j > i}} Pairwise Energy .$$

148. The system of claim 147, wherein dead-end elimination comprises elimination of a side chain rotamer r, of residue i, when the inequality
$${E}_{{i}_{r}}^{template} - {E}_{{i}_{r}}^{template} + {\sum }_{j \neq i} {min}_{s} ( {E}_{{i}_{r} - {j}_{s}}^{pairwise} - {E}_{{i}_{r} {j}_{s}}^{pairwise} ) > 0$$
is true.

149. The system of claim 122, further comprising the step of eliminating a side chain rotamer pair using dead-end elimination, wherein a side chain rotamer pair comprises a first side chain rotamer representing a first building block in said set of building blocks and a second side chain rotamer representing a second building block in said set of building blocks.

150. The system of claim 149, wherein dead-end elimination comprises eliminating a side chain rotamer pair that consists of rotamer r, of residue i, and rotamer s, of residue j, when the inequalities:
$${E}_{( {i}_{r} , {j}_{s} )} + {\sum }_{k \neq i , j} {min}_{t} ( {E}_{( {i}_{r} , {j}_{s} ) , {k}_{t}} ) > {E}_{( {i}_{u} , {j}_{v} )} + {\sum }_{k \neq i , j} {max}_{t} ( {E}_{( {i}_{u} , {j}_{v} ) , {k}_{t}} ) and$$
$${E}_{( {i}_{r} , {j}_{s} )} - {E}_{( {l}_{u} , {j}_{v} )} + {\sum }_{k \neq i , j} {min}_{i} ( {E}_{( {i}_{r} , {j}_{s} ) , {k}_{i}} - {E}_{( {i}_{u} , {j}_{v} ) , {k}_{t}} ) > 0$$
and
are true.

151. The system of claim 147, wherein the eliminating step is repeated until no additional side chain rotamers are eliminated from said array by the process of dead-end elimination.

152. The system of claim 149, wherein the eliminating step is repeated until no additional side chain rotamer pairs are eliminated from said array by the process of dead-end elimination.

153. The system of claim 122, wherein the solution score additionally comprises an entropy term that is determined by a difference between (i) an entropy of a side chain rotamer in the solution when substituted into the atomic structure and (ii) an entropy of the side chain rotamer in the solution when in a denatured state.

154. The system of claim 153, wherein entropy contributions of said side chain rotamers are derived from experimental or empirical data.

155. The system of claim 153, wherein the entropy term is computed using an iterative method.

156. The system of claim 153, wherein the entropy term is computed using mean field theory.

157. The system of claim 154, wherein the contribution of the rotamers to the entropy may be split into intrinsic and pairwise terms.

158. The method of claim 1 additionally comprising the step of synthesizing at least one of said solution structures which has a calculated solution score lower than said threshold value.

159. The method of claim 158 additionally comprising the step of screening each of said solution structures that has been synthesized against an assay to test for activity.

160. The method of claim 1 wherein at least one of said building blocks in said set of building blocks contacts a binding partner of said target macromolecule when bound to said target macromolecule.
